(19) United States
(12) Patent Application Publication
(10) Pub. No.: US 2015/0057512 A1
(43) Pub. Date: Feb. 26, 2015

(54) Wearable heart failure monitor patch

(71) Applicant: Rijuven Corporation, Wexford, PA (US)

(72) Inventor: Rajeshwar Kapoor, Pittsburgh, PA (US)

(21) Appl. No.: 14/121,307

(22) Filed: Aug. 18, 2014

Related U.S. Application Data
(63) Continuation-in-part of application No. 13/694,594, filed on Dec. 14, 2012, now Pat. No. 8,855,757.
(60) Provisional application No. 61/629,318, filed on Nov. 16, 2011.

(51) Int. Cl. A61B 5/0205; A61B 5/0456; A61B 5/08; A61B 5/00; A61B 7/04; A61B 5/1455

(52) U.S. Cl. CPC A61B 5/0205; A61B 7/04; A61B 5/14552; A61B 5/08; A61B 5/4818; A61B 5/0456
USPC 600/324; 600/513; 600/484

(57) ABSTRACT

The invention is directed to a system for acquiring electrical footprint of the heart, electrocardiogram (EKG or ECG), heart sound, heart rate, nasal airflow and pulse oximetry incorporated into a mobile device accessory. The ECG and heart sound signals are conveniently acquired and transmitted to a server via the mobile device, offering accurate heart failure analysis, and sleep disorder breathing indication.

### REFERENCE TO CO-PENDING APPLICATION(S)

The present application is a continuation in part of U.S. Non-Provisional patent application Ser. No. 13/694594, filed on Dec. 14, 2012 which is related to U.S. Provisional Patent Application Ser. 61/629318 filed on Nov. 16, 2011, the entire disclosures of which are hereby incorporated by reference.

### TECHNICAL FIELD

The present invention relates to the field of portable devices and more particularly to portable devices comprising a biometric sensor arrangement for measuring one or more intrinsic physical or health characteristic of a human.

### BACKGROUND OF THE INVENTION

The autonomic nervous system (ANS) regulates “involuntary” organs, while the contraction of voluntary (skeletal) muscles is controlled by somatic motor nerves. Examples of involuntary organs include respiratory and digestive organs, and also include blood vessels and the heart. Often, the ANS functions in an involuntary, reflexive manner to regulate glands, to regulate muscles in the skin, eye, stomach, intestines and bladder, and to regulate cardiac muscle and the muscle around blood vessels, for example.

The ANS includes the sympathetic nervous system and the parasympathetic nervous system. The sympathetic nervous system is affiliated with stress and the “fight or flight response” to emergencies. Among other effects, the “fight or flight response” increases blood pressure and heart rate to increase skeletal muscle blood flow, and decreases digestion to provide the energy for “fighting or fleeing.” The parasympathetic nervous system is affiliated with relaxation and the “rest and digest response” which, among other effects, decreases blood pressure and heart rate, and increases digestion to conserve energy. The ANS maintains normal internal function and works with the somatic nervous system. Afferent nerves convey impulses toward a nerve center, and efferent nerves convey impulses away from a nerve center.

The heart rate and force is increased when the sympathetic nervous system is stimulated, and is decreased when the sympathetic nervous system is inhibited (the parasympathetic nervous system is stimulated). Cardiac rate, contractility, and excitability are known to be modulated by centrally mediated reflex pathways. Baroreceptors and chemoreceptors in the heart, great vessels, and lungs, transmit cardiac activity through vagal and sympathetic afferent fibers to the central nervous system. Activation of sympathetic afferents triggers reflex sympathetic activation, parasympathetic inhibition, vasoconstriction, and tachycardia. In contrast, parasympathetic activation results in bradycardia, vasodilation, and inhibition of vasopressin release. Among many other factors, decreased parasympathetic or vagal tone or increased sympathetic tone is associated with various arrhythmias genesis, including ventricular tachycardia and atrial fibrillation.

Stimulating the sympathetic and parasympathetic nervous systems can have effects other than heart rate and blood pressure. For example, stimulating the sympathetic nervous system dilates the pupil, reduces saliva and mucus production, relaxes the bronchial muscle, reduces the successive waves of involuntary contraction (peristalsis) of the stomach and the motility of the stomach, increases the conversion of glycogen to glucose by the liver, decreases urine secretion by the kidneys, and relaxes the wall and closes the sphincter of the bladder. Stimulating the parasympathetic nervous system (inhibiting the sympathetic nervous system) constricts the pupil, increases saliva and mucus production, contracts the bronchial muscle, increases secretions and motility in the stomach and large intestine, and increases digestion in the small intention, increases urine secretion, and contracts the wall and relaxes the sphincter of the bladder. The functions associated with the sympathetic and parasympathetic nervous systems are many and can be complexly integrated with each other.

Neural stimulation can be used to stimulate nerve traffic or inhibit nerve traffic. An example of neural stimulation to stimulate nerve traffic is a lower frequency signal (e.g. within a range on the order of 20 Hz to 50 Hz). An example of neural stimulation to inhibit nerve traffic is a higher frequency signal (e.g. within a range on the order of 120 Hz to 150 Hz). Other methods for stimulating and inhibiting nerve traffic have been proposed. According to various embodiments of the present subject matter, sympathetic neural targets include, but are not limited to, a peroneal nerve, a sympathetic column in a spinal cord, and cardiac post-ganglionic sympathetic neurons. According to various embodiments of the present subject matter, parasympathetic neural targets include, but are not limited to, a vagus nerve, a baroreceptor, and a cardiac fat pad. Neural stimulation can be selectively delivered to afferent neural pathways, selectively delivered to efferent neural pathways, or delivered to both afferent and efferent neural pathways. For example, some embodiments selectively stimulate or inhibit only parasympathetic afferents or only parasympathetic efferent, and some embodiments selectively stimulate or inhibit sympathetic afferents or efferent.

The present subject matter can be used to prophylactically or therapeutically treat various diseases by modulating autonomic tone. Examples of such diseases or conditions include hypertension, cardiac remodeling, and heart failure.

Hypertension is a cause of heart disease and other related cardiac co-morbidities. Hypertension occurs when blood vessels constrict. As a result, the heart works harder to maintain flow at a higher blood pressure, which can contribute to heart failure. Hypertension generally relates to high blood pressure, such as a transitory or sustained elevation of systemic arterial blood pressure to a level that is likely to induce cardiovascular damage or other adverse consequences. Hypertension has been defined as a systolic blood pressure above 140 mm Hg or a diastolic blood pressure above 90 mm Hg. Consequences of uncontrolled hypertension include, but are not limited to, retinal vascular disease and stroke, left ventricular hypertrophy and failure, myocardial infarction, dissecting aneurysm, and renovascular disease. A large segment of the general population, as well as a large segment of patients implanted with pacemakers or defibrillators suffer from hypertension. The long term mortality as well as the quality of life can be improved for this population if blood pressure and hypertension can be reduced. Many patients who suffer from hypertension do not respond to treatment, such as treatments related to lifestyle changes and hypertension drugs.

Following myocardial infarction (Ml) or other cause of decreased cardiac output, a complex remodeling process of the ventricles occurs that involves structural, biochemical, neurohormonal, and electrophysiologic factors. Ventricular remodeling is triggered by a physiological compensatory mechanism that acts to increase cardiac output due to so-called backward failure which increases the diastolic filling pressure of the ventricles and thereby increases the so-called preload (i.e., the degree to which the ventricles are stretched by the volume of blood in the ventricles at the end of diastole). An increase in preload causes an increase in stroke volume during systole, a phenomena known as the FrankStarling principle. When the ventricles are stretched due to the increased preload over a period of time, however, the ventricles become dilated. The enlargement of the ventricular volume causes increased ventricular wall stress at a given systolic pressure. Along with the increased pressure-volume work done by the ventricle, this acts as a stimulus for hypertrophy of the ventricular myocardium. The disadvantage of dilatation is the extra workload imposed on normal, residual myocardium and the increase in wall tension (Laplace's Law) which represent the stimulus for hypertrophy. If hypertrophy is not adequate to match increased tension, a vicious cycle ensues which causes further and progressive dilatation. As the heart begins to dilate, afferent baroreceptor and cardiopulmonary receptor signals are sent to the vasomotor central nervous system control center, which responds with hormonal secretion and sympathetic discharge. It is the combination of hemodynamic, sympathetic nervous system and hormonal alterations (such as presence or absence of angiotensin converting enzyme (ACE) activity) that ultimately account for the deleterious alterations in cell structure involved in ventricular remodeling. The sustained stresses causing hypertrophy induce apoptosis (i.e., programmed cell death) of cardiac muscle cells and eventual wall thinning which causes further deterioration in cardiac function. Thus, although ventricular dilation and hypertrophy may at first be compensatory and increase cardiac output, the processes ultimately result in both systolic and diastolic dysfunction (decompensation). It has been shown that the extent of ventricular remodeling is positively correlated with increased mortality in post-MI and heart failure patients.

Heart failure refers to a clinical syndrome in which cardiac function causes a below normal cardiac output that can fall below a level adequate to meet the metabolic demand of peripheral tissues. Heart failure may present itself as congestive heart failure (CHF) due to the accompanying venous and pulmonary congestion. Heart failure can be due to a variety of etiologies such as ischemic heart disease. Heart failure patients have reduced autonomic balance, which is associated with LV dysfunction and increased mortality. Modulation of the sympathetic and parasympathetic nervous systems has potential clinical benefit in preventing remodeling and death in heart failure and post-MI patients. Direct electrical stimulation can activate the baroreflex, inducing a reduction of sympathetic nerve activity and reducing blood pressure by decreasing vascular resistance. Sympathetic inhibition and parasympathetic activation have been associated with reduced arrhythmia vulnerability following a myocardial infarction, presumably by increasing collateral perfusion of the acutely ischemic myocardium and decreasing myocardial damage.

The prior art teaches many way of measuring heart rate. A stethoscope is traditionally used to amplify these sounds and present them to a caregiver. The acoustic principle may also be used in other ways, both manual and automated, at various parts of the body. Another way is the pulse oximeter approach. In pulse oximeters, a light of a known frequency through an area of the body, such as the fingertip or earlobe, and detect the same light once it has either passed through the body or been reflected back to a photo sensor. With each heartbeat, oxygen-rich blood is momentarily pushed through the capillaries in that region. This momentary increase in the oxygen content of the blood upon each heart beat changes the optical properties of the blood. As the light passes through the fingertip or earlobe, specific frequencies are absorbed to varying degrees, depending on the amount of oxygen in the blood, and are therefore not present in the returning light. The change in detected frequencies occurring once per heart beat allows for detection of individual heart beats, and thus a heart rate measurement. The degree of spectral change is used to determine the oxygen content in the blood. Another measurement method makes use of the varying outward pressure applied against the skin by major arteries. With each heartbeat, a surge of blood passes through the arteries. In an artery of sufficient size, and located near to the surface of the body, this momentary pressure can be detected by holding a pressure sensor, such as a piezo-electric (P-E) element, in place over the artery location. The P-E element is physically stretched by the momentary outward pressure of the artery during a heart-beat. As it is stretched, the altered shape of the P-E element changes its electrical characteristics--e.g., a change in its resistance to a current passing through it. Changes in the resistance of the P-E are then detected by appropriate circuitry, and used to identify heart beats and thus heart rate. Suitable surface arteries and sensing devices are well known in the art and include sensing at the wearer's wrist, the temple, the inner ear, or the bridge of the nose.

Heart rate monitoring using the chest strap method has become increasingly popular for sports and fitness training as well as for some other activities such as relaxation training, stress relief and meditation in which heart rate as a bio-feedback item has been found useful. During this time, the chest strap has remained in much the same form, as a practical means of obtaining a continuous, accurate heart rate reading for these largely non-medical purposes. However, for many users, the chest strap may chafe causing discomfort. Many users find them awkward to put on, uncomfortable to wear, and bothersome to keep handy. In addition, they can be restrictive of good chest expansion and thus restrict full breathing during exercise. For wearers with slender ribs and torsos, the chest strap can slip down out of the proper position and cease to function properly. Stretched across the chest, they are perceived by some as unmanly, or unwomanly, or as interfering with tan lines or undergarments.

There are various physiological factors affecting the autonomic regulation of heart rate: respiration, thermoregulation, hormonal regulation, blood pressure, cardiac output, etc. One of the most important factors is blood pressure. There are special cells in the heart and large blood vessels that sense blood pressure level and send afferent stimulation to the central structures of the ANS that control HR and blood vessel tonus forming a continuous feedback to maintain an optimal level of the blood pressure.

This mechanism is also called baroreflex. It increases HR when blood pressure drops and vice versa and thus maintains a short-term stable blood supply to the vital organs.

One of the best ways to assess the autonomic function is to analyze minute changes in heart rate, which are caused by many factors including regulatory influence of the autonomic nervous system.

A special method of analysis can be applied to recorded heart rate readings. It is called Heart Rate Variability (HRV) analysis. The HRV analysis is a powerful, very accurate, reliable, reproducible, yet simple to do.

It is found that lowered HRV is associated with aging, decreased autonomic activity, hormonal tonus, specific types of autonomic neuropathies (e.g. diabetic neuropathy) and increased risk of sudden cardiac death after acute heart attack.

Other research indicated that depression, panic disorders and anxiety have negative impact on autonomic function, typically causing depletion of the parasympathetic tonus. On the other hand an increased sympathetic tonus is associated with lowered threshold of ventricular fibrillation. These two factors could explain why such autonomic imbalance caused by significant mental and emotional stress increases risk of heart attack followed by sudden cardiac death.

Aside from that, there are multiple studies indicating that HRV is quite useful as a way to quantitatively measure physiological changes caused by various interventions both pharmacological and non-pharmacological during treatment of many pathological conditions having significant manifestation of lowered HRV.

However it is important to realize that clinical implication of HRV analysis has been clearly recognized as a predictor of risk of arrhythmic events or sudden cardiac death after acute heart attack, and as clinical marker of diabetic neuropathy evolution.

Nevertheless, as the number of clinical studies involving HRV in various clinical aspects and conditions grows, HRV remains one of the most promising methods of investigating general health in the future.

There is an ongoing need for an improved system and method for heart rate, heart rate variability, wellness and fitness monitoring that is user friendly and less invasive.

Heart Failure—Congestive heart failure (CHF) is defined as impairment of systolic and/or diastolic function of the heart, leading to failure to meet the demands of peripheral tissues, or leading to maintenance of cardiac function under higher filling pressures.

In other words, CHF is a complex clinical entity that is characterized by ventricular dysfunction and compensatory neuron-hormonal alteration accompanied by exercise intolerance, fluid retention, and decreased life expectancy.

In CHF, the renin-angiotensin-aldosterone system and the sympathetic nervous system are considered to be of the utmost importance. These 2 interrelated systems regulate vascular tonus, heart rate, and contractility. CHF may stem from diastolic or systolic dysfunction.

In general, the 5-y mortality rate has been around 75% for men and 65% for women with CHF, whereas the 1-y mortality rate for severe and moderate forms of the disease reaches 40% to 50% and 15% to 25%, respectively. Coexistence of systolic and diastolic dysfunction is a frequent finding in CHF. Systolic dysfunction, in particular, is considered to be much more important in predicting the morbidity and mortality rates of the disease.

Echocardiographic evaluation of left ventricular ejection fraction (LVEF) obtained via the Simpson method has been an important parameter in the evaluation of systolic function, whereas atria-ventricular diastolic inflow waves have been obtained to assess diastolic function of the heart. Myocardial performance index (MPI) (Tei index) has been regarded as an important parameter in the evaluation of ventricular systolic function in congestive heart failure. MPI is defined as the sum of isovolumic relaxation time (IVRT) and isovolumic contraction time (IVCT) divided by left ventricular ejection time (LVET) ([IVRT+IVCT]/LVET). MPI is a Doppler-derived time interval index that combines both systolic and diastolic cardiac performance. The Tei index has been difficult to derive by using conventional non-Doppler pulsed echocardiography.

Sleep Apnea—Obstructive Sleep Apnea (OSA) is a common medical disorder with serious medical, psychological, and behavioral consequences (Aloia, Arnedt, Davis, Riggs, & Byrd, 2004; Ancoli-Israel & Kripke, 1991; Nieto et al., 2000; Young et al., 1993). OSA is evident in 2% to 4% of middle-aged women and men, respectively (Young et al., 1993), with increased prevalence in elderly populations (Ancoli-lsrael & Kripke, 1991) and in certain racial groups (Redline et al., 1997). Daytime consequences of OSA include excessive daytime sleepiness; mood changes such as depression and irritability; and subjective impairments in cognitive domains such as attention, concentration, and memory. Other co-morbidities include hypertension (high blood pressure), heart failure, coronary artery disease, obesity, stroke and sudden death. These consequences have a negative effect on the quality of life of OSA sufferers. Despite recent advances in physician recognition of the disorder, a large proportion of OSA patients remain undiagnosed and untreated. This is particularly troubling in light of the associations between undiagnosed obstructive sleep apnea and chief causes of morbidity and mortality in adults (Nieto et al., 2000; Shahar et al., 2001).

Although recognition of the disorder has increased in recent years, the majority of OSA patients remain undiagnosed and untreated, which is particularly troubling in light of associations between undiagnosed sleep apnea and chief causes of morbidity and mortality (Nieto et al., 2000; Shahar et al., 2001). Effective treatments exist for OSA, the most efficacious being positive airway pressure (PAP). Several randomized clinical trials have demonstrated the superiority of PAP to placebo and conservative treatments for treating respiratory disturbances and for improving the daytime sequelae associated with OSA (Engleman et al., 2002; Montserrat et al., 2001; Redline et al., 1998). Despite its efficacy, it is estimated that over 50% of those started on CPAP will not be using it 1 year later (Loube et al., 1999). Of those patients using it 1 year later, most are not using CPAP for the entire night as prescribed (Published adherence rates from studies in the United States range from 3.3 to 5.3 hr per night (Kribbs et al., 1993; Reeves-Hoche, Meck, & Zich, 1994; Stepnowsky, Marler, & Ancoli-Israel, 2002). One study showed that only 6% of patients use the device for hr or more per night on at least 70% of the nights (Kribbs et al., 1993). Symptomatic improvements may result with average nightly adherence of 4.5 hr; however, alertness is impaired even with a single missed night of treatment (Kribbs et al., 1993a), negatively impacting quality of life. The Importance of regular adherence is highlighted by a recent longitudinal study that demonstrated a higher 5-year survival rate for adherent users of treatment compared with nonusers (Campos-Rodriguez et al., 2005).

More than 20 million Americans suffer from OSA. However, up to 93% of women and 82% of men with OSA have yet to be diagnosed (Young T, Evans L, Finn L, et al. Estimation of the clinically diagnosed proportion of sleep apnea syndrome in middle-aged men and women. Sleep. 1997; 20:705-706.). The market has grown 15-25% annually for the past five years, fueled by increased awareness of the disease (Counting Sleep BYLINE: BY DANISH H. MCCLINTON—December 2006, Homecare Magazine). In 2008, over $4.3 billion was spent on the diagnosis and treatment of OSA (Counting Sleep BYLINE: BY DANISH H. MCCLINTON—December 2006, Homecare Magazine). By 2015, the worldwide market is projected to reach $7.4 billion).

The current model for diagnosing and treating OSA is expensive, uncomfortable and time consuming. The model is fragmented (i.e. going one place for diagnosis and another for treatment), which can be frustrating for patients and their doctors. Additionally, access to care is severely limited, with sleep labs backlogged nationwide. Once diagnosed, OSA patients remain one of the most difficult groups to care for, as over 54% (Kribbs NB, Pack Al, Kline LR, et al. Objective measurement of patterns of nasal CPAP use by patients with obstructive sleep apnea. Am Rev Respir Crit Care Dis. 1993; 147:887-95) of patients do not remain compliant with positive airway pressure (PAP) therapy, the gold standard treatment for OSA. The most common reasons cited for non-compliance include; a gross lack of information about therapy, and products that don't meet customer's needs.

The complexities of the existing system are compounded by the fact that OSA is both deadly and costly. OSA increases the risk of premature death by 46% among severe sufferers (Johns Hopkins Medical Center, July 2009). Yearly, there are over 38,000 OSA-related cardiovascular deaths (National Commission of Sleep Disorder Research) and over 1,400 deaths due to OSA-related automobile accidents in the U.S. (Findley et al. Reduction in motor vehicle collision cost and fatalities in OSA sufferers, Sleep Magazine 2004). The medical cost of undiagnosed OSA is $3.4 billion per year (Kapur et al. The Medical Cost of Undiagnosed Sleep Apnea, 1999). OSA related motor vehicle accidents represent a cost of $16 billion per year in the U.S.

### SUMMARY OF THE INVENTION

One object of the invention is to provide a mobile system that monitors the electrical conductivity and the mechanical function of the heart for analyzing the entire cardiac cycle, wherein a user can track electrical deficiencies of the heart along with any abnormalities of the cardiac cycle, thereby detecting heart failure.

A second aspect of the invention is to provide a mobile system that monitors the cardiac cycle by analyzing the electrical conductivity and mechanical functions of the heart for heart rate. The mobile system measures the isovolumic contraction time (IVCT), isovolumic relaxation time (IVRT) and ejection time (ET), wherein a user abnormalities to the cardiac cycle that may be surrogate to heart failure and ejection fraction.

A third aspect of the invention is to provide a mobile system that collects data such as ECG, heart sound, nasal air pressure, actigraphy rest/activity cycles to determine sleep patterns, body temperature, EEG, heart rate, heart rate variability, fall detection, gait analysis. pulse oximetry (blood oxygen—SaO2 or SPO2 and heart rate) data. The sensor device can also measure blood pressure and skin temperature. The sensor device also monitors data such as breathing cessation or apnea-hypopnea index (AHI). The combined data such as AHI, SPO2, heart rate and body movement are used to diagnose and monitor obstructive sleep apnea OSA.

The invention includes a method and system for administering a diagnosis of patients suffering from sleep disordered breathing conditions comprising: determining prevalence of sleep disordered breathing and co-morbidities; collecting at least blood oxygen saturation, heart rate, nasal airflow, sleep staging and body movement data from a sensor device; transmitting data collected from the sensor device to a remote health management server; diagnosing sleep disordered breathing using the data collected from the sensor device; communicating diagnosis information to a client device;

### DETAILED DESCRIPTION:

### BRIEF DESCRIPTION OF THE DRAWINGS

The present invention will now be described in more detail with reference to the enclosed drawings, in which:

FIG. 1 shows a block diagram of the overall system comprising of a mobile ECG system a mobile device and backend server.

FIG. 2 shows a flow diagram of the mobile ECG system.

FIG. 3 shows an ECG recorder in the form of a mobile case.

FIG. 4 shows another view of the ECG recorder in the form of a mobile case.

FIG. 5 shows an ECG recorder in the form of headphone and upper arm band.

FIG. 6 shows an Arm Strap/band with sensor opening.

FIG. 7 shows a version of the ECG recorder in the form of a headphone.

FIG. 8 shows shows a headphone jack that works in conjunction with the headphone.

FIG. 9 shows exemplary health information modules that are being communicated to the user as a result of heart rate variability analysis.

FIG. 10 shows a version of the ECG recorder in the form of a chest patch.

FIG. 11 shows an overall depiction of the system and how they are connected.

FIG. 12 shows the inter-connectivity of the devices.

FIG. 13 shows the component of the sensor device.

FIG. 14 shows an algorithm of acquiring systolic performance index.

FIG. 15 shows a depiction of the cardiac cycle.

FIG. 16 shows a sequence of the major components.

FIG. 17 shows XYZ coordinates of the accelerometer.

### DETAILED DESCRIPTION

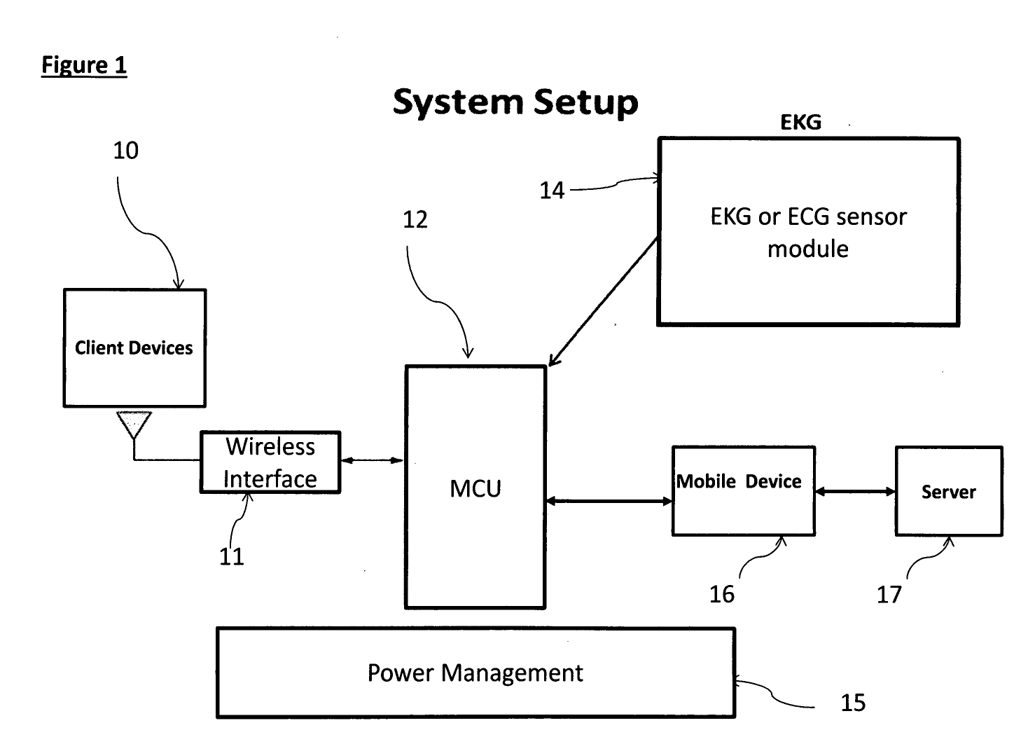

The inventive system, in accordance to FIG. 1, consists of an electrical footprint of the heart or ECG recorder device. The ECG recorder device consists of a microprocessor 12, power management 15, dry ECG sensors and processor 14. The ECG recorder includes wireless interface 11 capable of communicating with client devices 10 such as a mobile device 16. The mobile device includes software component running on it, interfacing the ECG recorder with a back-end server 17, with the capability to capture and save ECG data, and analyzes it to obtain a marker for heart rate variability.

In a preferred embodiment, the ECG recorder interfaces with the mobile or cellular phone via Bluetooth wireless communication protocol to mobile device 16. The ECG recorder can also connect with the mobile device 16 via wireless local area network (WLAN) products that are based on the Institute of Electrical and Electronics Engineers' (IEEE) 802.11 standards such as Wi-Fi.

Customized software application is installed on the mobile device 16 to review physiologic data of a patient and to (a) view the near real-time waveforms remotely (b) remotely review other standard patient data. The customized software can display at least the following physiologic information: ECG Waveform, health assessment metrics such as Overall Health and Wellness, Biological clock, Fitness Level and Stress Level.

The CPU processor 12 comprises a tangible medium, for example read only memory (ROM), electrically erasable programmable read only memory (EEPROM) and/or random access memory (RAM). The processor 12 may also be comprised many known real time clock and frequency generator circuitries, for example the PIC series of processors available from Microchip, of Chandler Ariz. In some embodiments, processor may comprise the frequency generator and real time clock.

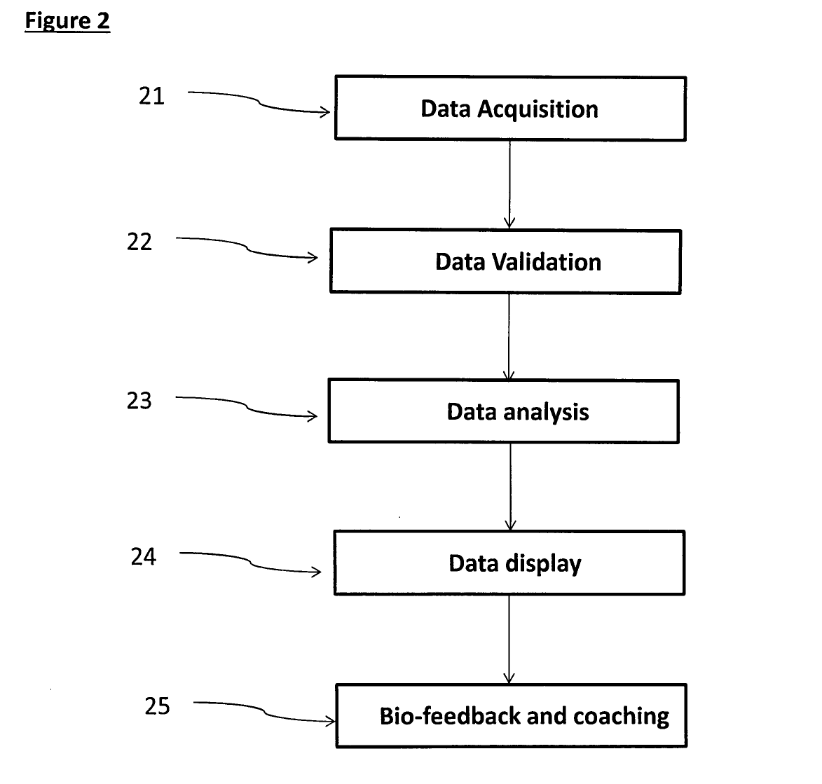

In FIG. 2, the ECG recorder acquires heart electrical footprint or ECG data from the user 21. The ECG data gets sent to the mobile device 16, where it gets validated 22. The mobile device sends the ECG data to the backend server for further analysis 23. After analysis, the backend server sends the health module information to the mobile 16 where it gets displayed 24 for biofeedback and coaching 25.

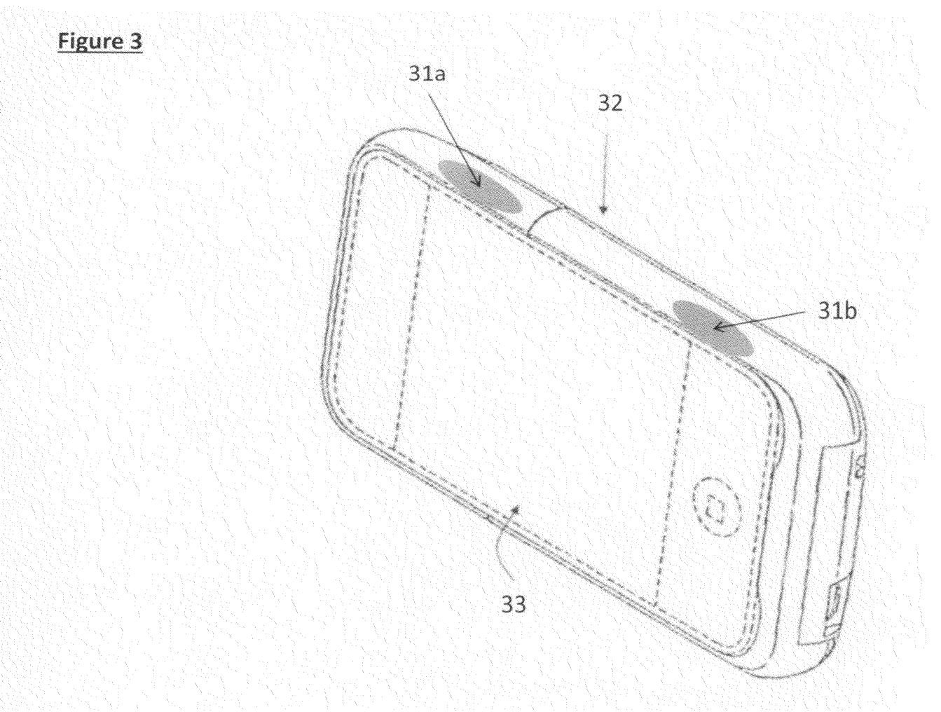

FIG. 3 shows an exemplary ECG recorder in the form of a mobile cover case 32 (also referred to as “cover”). The cover 32 can also be a mechanism that partially covers the mobile device 33. The cover 32 encompasses at least two electrodes 31a-b, on its sides or back to establish contact with the user's fingers and/or chest. The electrodes can be made out of stainless steel, silicon nitride and silver-silver chloride, with dimensions between approximately 4 mm to about 10 mm in diameter or in width and height. The two electrodes 31a-b (positive and negative) may be paired with a third electrode to serve as a reference voltage (ground) for the differential amplifier and to improve common mode noise rejection.

Holding the cover in one hand, e.g., the left hand, the patient makes contact with one electrode, one on one side of the cover which contacts the thumb. The patient then touches the other side of the cover with the other hand, making contact with the other electrode to the other side. ECG and heart rate are thereby recorded for 1 to 5 minutes.

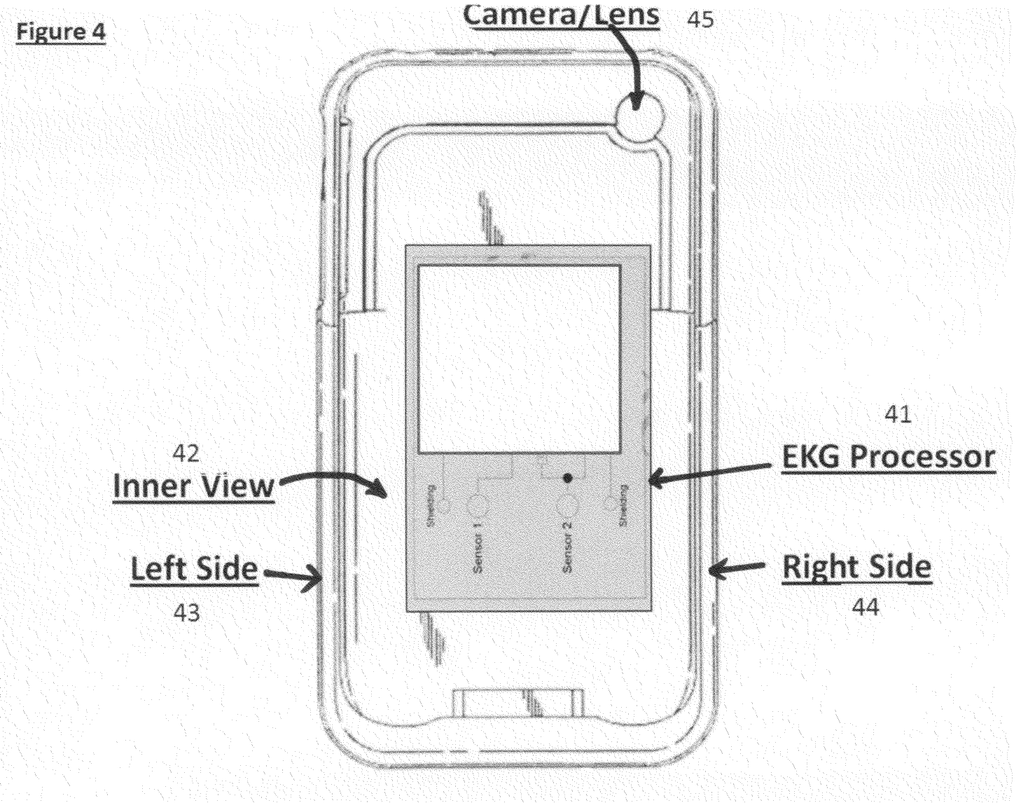

In FIG. 4, the cover contains electrodes are attached to a microprocessor 41 (MCU), performing bio-signal detection and processing. The MCU 41 is designed with advanced analog front end circuitry and a flexible, powerful digital signal processing structure. It targets bio-signal inputs ranging from uV to mV level and deployed with proprietary algorithms. The Low-Noise-Amplifier and ADC are the main components of the MCU 41 analog front end. It can detect bio-signals and convert them into digital words using a 16-bit high resolution analog digital converter (ADC). The heart of the MCU 41 digital circuit is a powerful system management unit. It is in charge of overall system configuration, operation management, internal/external communication, proprietary algorithm computation, and power management. The MCU 41 also comes with hardwired DSP blocks to accelerate calculations, such as various digital filtering, under the supervision of the system management unit. In other embodiments, the cover 33 is used as a Mobile Heart Rate Monitor for regular and long term usage for applications such as heart rhythm irregularity detection.

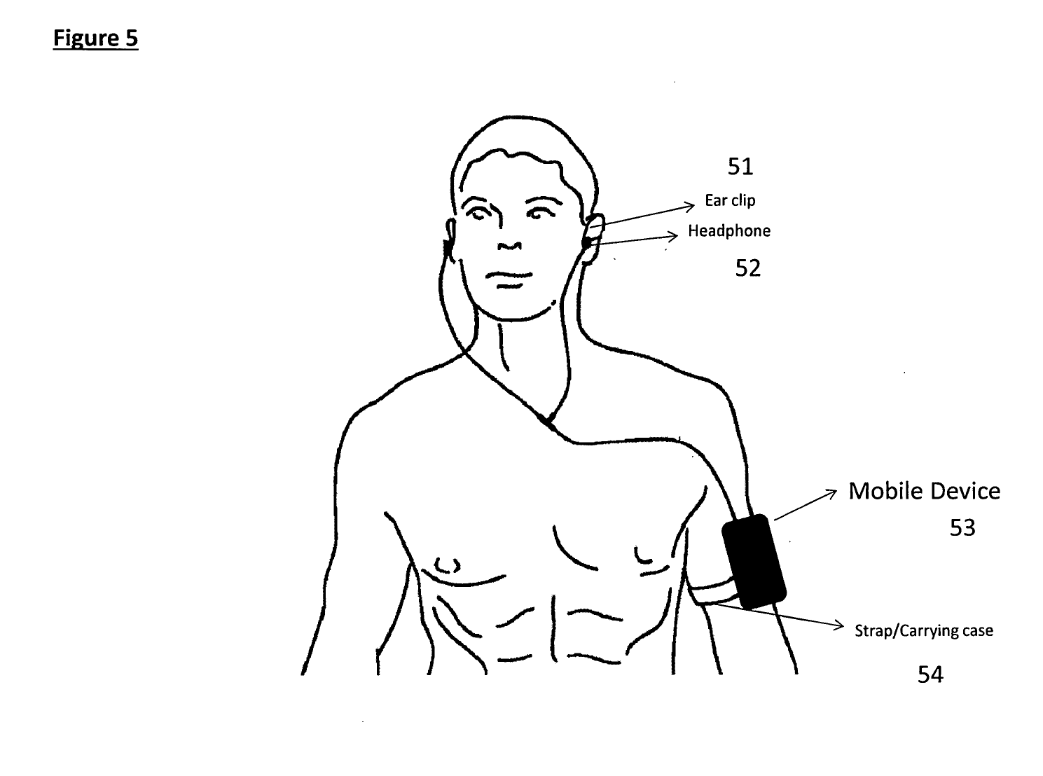

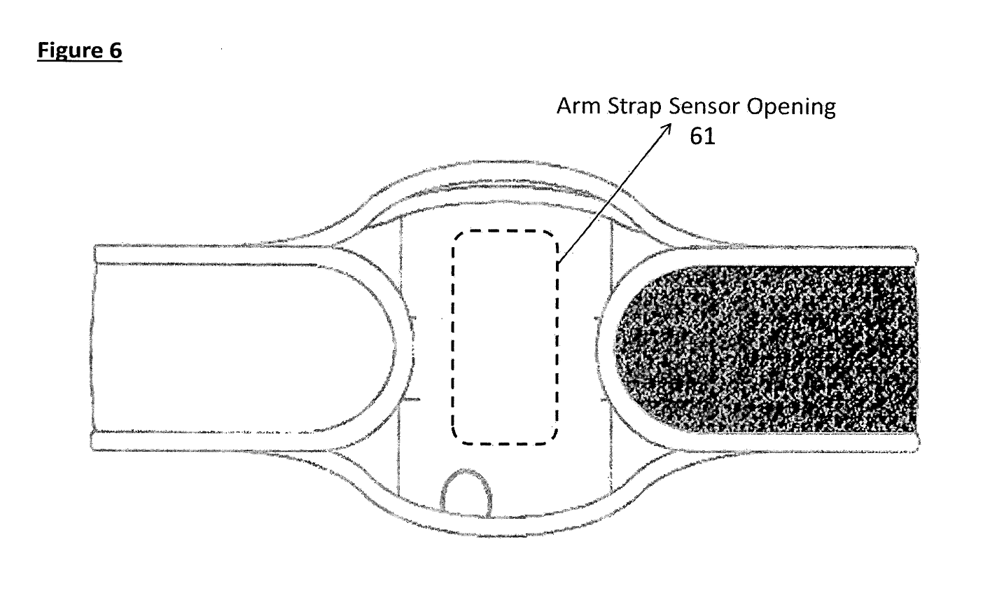

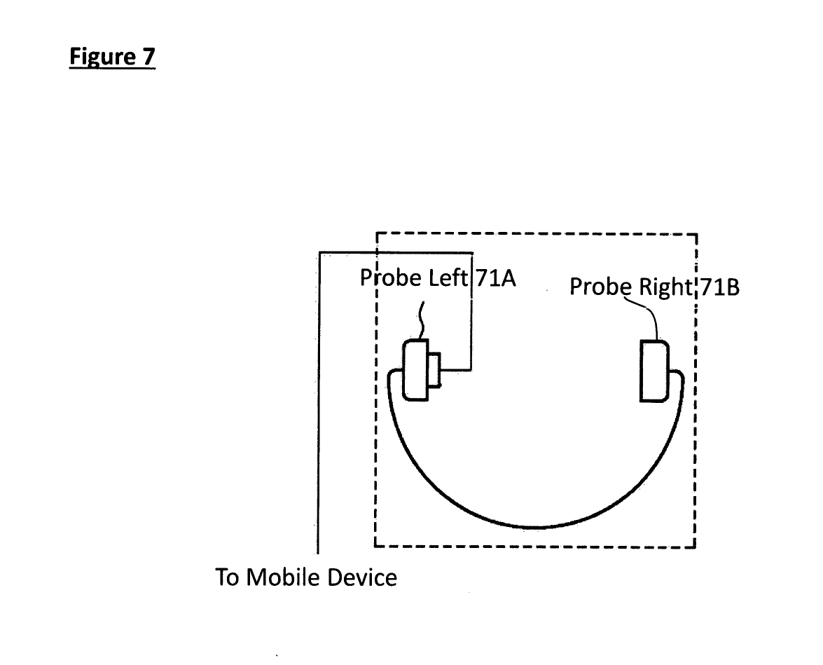

According to one aspect shown in FIG. 5, the invention can be realized as a specially designed headphone 51, 52 and left extremity strap 54. The invention encompasses at least two electrodes on the inner or outer right ear (hereafter referred to as ear) and another on the left extremity. The electrodes are made out of stainless steel, silicon nitride or silver-silver chloride, with dimensions between approximately 3 mm to 10 mm in diameter or in width/length. The two electrodes in FIG. 7, 71a and b (positive and negative) are paired with a third electrode to serve as a reference voltage (ground) for the differential amplifier and to improve common mode noise rejection. The preferred system features a negative electrode on the right ear and a positive or ground electrode on a strap FIG. 6, 61, touching the skin on the lower arm.

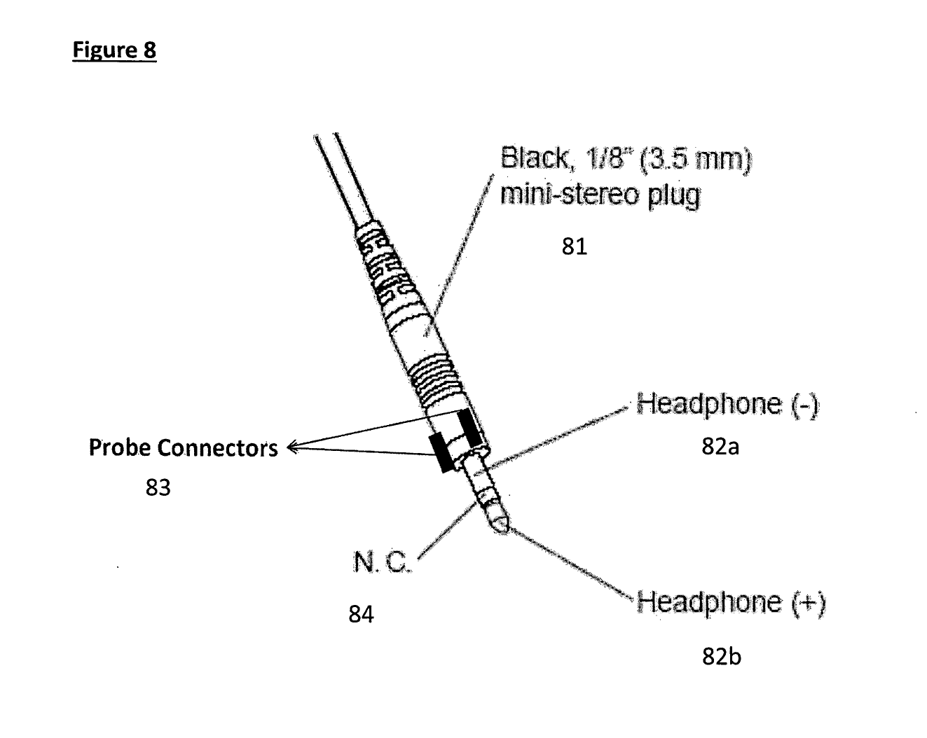

According to FIG. 8, wires connect these electrodes are mated with the main ECG recorder device via a specialized headphone jack. The Jack includes a mini stereo plug 81. The jack also includes electrical connectors 84, making connection with the main recorder device, which has circuitry capable of measuring the electrical voltage, potential between the electrodes and to detect patterns therein corresponding to individual heart-beats and heart rate variability. EKG, heart rate and heart rate variability are thereby recorded for 1 to 5 minutes, and are reported to the user in various ways.

In another embodiment, the system has two electrodes FIG. 7, 71a-b, at least one electrode positioned to be in contact with the skin of the head, including the ear via a headphone or headset, and a second electrode positioned to be in contact either with the skin of the arm at the bicep (FIG. 5) or wrist, or else with the skin of the torso at the waist. The system serves as a headphone for listening to audio from an audio source device such as a portable MP3 player, a radio, a mobile telephone (e.g., cellular, portable, satellite, etc.), etc. An in-the-ear style of headphones, commonly known as “ear-buds”, can be used. Ear-buds are commonly worn one bud in each ear, such that the outer surface of each bud enclosure is in contact with the skin of the folds of the ear. In this embodiment, the outer surface of the bud enclosure is modified to be electrically conductive and made to serve as an electrode connected to the heart-beat detection circuitry. Some ear-bud designs, which are popular among exercisers, also contain a structure designed to fit around the ear, thus holding the ear-bud in place during vigorous physical activity. Such a design may also, in this embodiment, provide contact surfaces around the ear which may be used to hold a conductive surface (electrode) in constant contact with the skin around the ear.

The other electrode can be integrated into a carrying case used for carrying portable audio devices FIG. 6, 54. Exercisers who wish to wear a portable audio device (MP3 player, radio, mobile telephone, etc.) frequently wear the audio source device in one of several locations: strapped to the upper arm, strapped to the wrist or forearm, clipped to the waistband of exercise clothing, held in the hand, etc. Features and aspects hereof may include an apparatus which holds the portable audio source device and the heart rate detection circuit in one of those convenient locations and integrates a conductive surface at that location to serve as one of the required electrodes connected to the heart rate detection circuit.

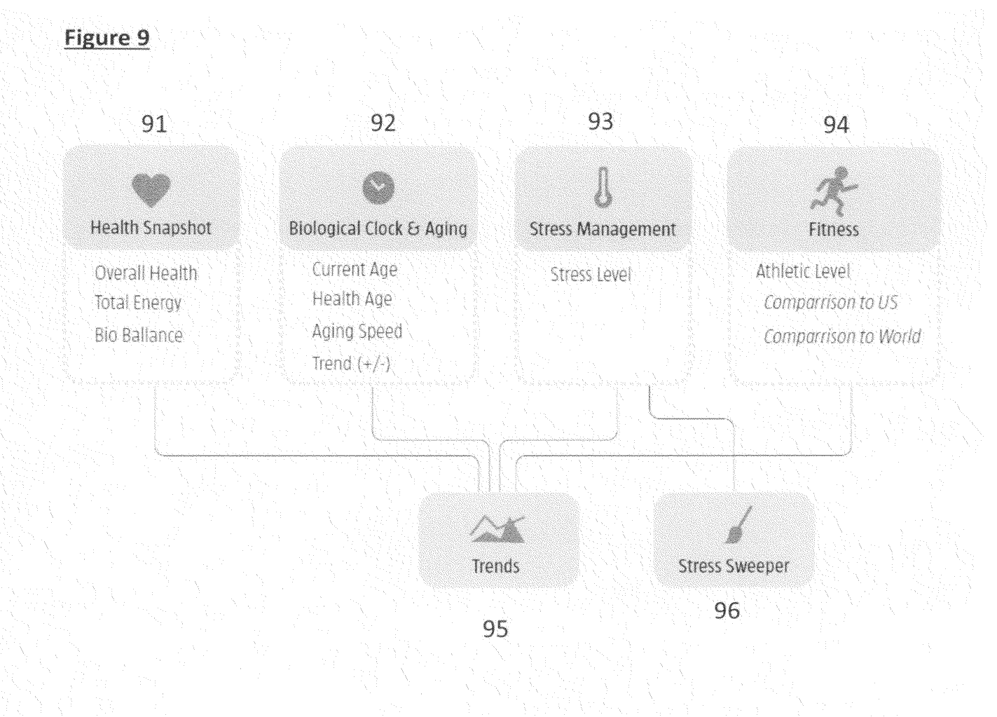

HRV is being used to derive health assessment metrics, FIG. 9 such as overall health and wellness 91, aging 92, fitness 94 and stress 93. The source information for HRV analysis is continuous beat-by-beat (not averaged) recording of heartbeat intervals. The electrical footprint of the heart or Electrocardiograph (ECG or EKG) is considered as the best way to measure heartbeat intervals. ECG is an electrical signal reflecting minute changes in the electrical field generated by heart muscle cells. It is measured by a special electronic device with conductive electrodes placed on chest around heart area or limbs. ECG signal has a very specific and robust waveform simple to detect and analyze. Cardiac rhythm (sequence of heartbeat intervals) derived from ECG is the best way to detect normal heartbeats as well as all sorts of ectopic heartbeats, which must be excluded from the HRV analysis.

The autonomic nervous system function can be evaluated with the Autonomic Balance Test. This test is based on the short-term HRV analysis of resting heart rate recordings of 1 to 5 minutes long. Such recordings are assumed to be done at a steady-state physiological condition and should be properly standardized to produce comparable results.

According to the standards set forth by the Task Force of the European Society of Cardiology and North American Society of Pacing and Electrophysiology in 1996, there are two methods of analysis of HRV data: time- and frequency-domain analysis. For both methods the heartbeat intervals should be properly calculated and any abnormal heartbeats found. HRV relates to the regulation of the sinoatrial node, the natural pacemaker of the heart by the sympathetic and parasympathetic branches of the autonomic nervous system. An HRV assessment is based on the assumption that the beat-to-beat fluctuations in the rhythm of the heart provide us with an indirect measure of heart health, as defined by the degree of balance in sympathetic and vagus nerve activity.

The ECG recorder collects ECG data from users which gets analyzed and converted to HRV data the gives information about different states of the Autonomic Nervous System. It happens that ANS states vary from one individual to another, especially of different age and gender. The backend server 17 matches the state of the user's Autonomic Nervous System to the range of “healthy” states of individuals of within the age range and gender. The correct results about your health 91 and fitness 94 are determined.

Fitness assessment 94 is based on the one of the most accurate methods of fitness assessment. Analysis is done on ECG signal at the backend server that determines the autonomic nervous system's response on a simple standup maneuver. The standup maneuver causes heart rate to rise within the first 10-15 seconds because blood pressure drops due to gravitational redistribution of the blood mass. Then the cardiovascular system attempts to compensate an orthostatic effect of standing up by constricting peripheral blood vessels. As a result blood pressure returns to its normal level and heart rate drops. Athlete body reaction is fast and strong, while sedentary lifestyle makes body react with a delay and a little amplitude. This serves as a base of determining a fitness level.

This biological clock 92 is determined based on the body's ANS (autonomic nervous system) response on paced breathing. The risk of myocardial infarction and overall health condition of the body is evaluated.

The current invention makes an assessment of the autonomic nervous system regulatory function condition based Autonomic Balance—a ratio between levels of the sympathetic and parasympathetic activity and Autonomic Tonus—a net level of the sympathetic and parasympathetic activity.

There are three main types of the autonomic nervous system conditions: 1-Predominant parasympathetic nervous system function—typical for a state of relaxation, 2-Predominant sympathetic nervous system function—typical for a state of stress and 3-Balanced autonomic nervous system function—typical for an idle calm state.

Each of these three categories may have three different levels of the autonomic tonus: low, normal or high. The Autonomic Balance is calculated in points based on 80% of least deviated values of HRV parameters in the normative database. It ranges from −10 points to +10 points.

To make a conclusion on the HRV analysis, actual ECG readings of all HRV parameters are compared with their respective normal ranges specific to patient's age and gender. These normal ranges are taken from a normative database built in a special clinical study on a large pool of clinically validated healthy subjects.

Normal range is a range of values of certain HRV parameter representing statistical distribution of this parameter values in a large population of healthy individuals of selected age and gender. For instance, the logarithmic value of HF (mŝ2/Hz) lies in range between 2.5 and 6.6 for males between 30 and 40 years old.

In this embodiment, a 48 years old male was tested with an autonomic balance test. The test results showed a Parameter value of 3.1 on logarithmic scale of mŝ2/Hz. A predicted value for 48 years old males is 4.03. One of the widely used approaches is determining a normal range based on criteria of statistical distribution of measured parameter values in healthy subjects. Typically normal range is considered within 95% of the interval of confidence in both directions. This range would fit 95% of all readings obtained from healthy subjects of the selected population. It is important to mention that there is a borderline zone (or conditional norm) in near proximity to the borders of the normal range. Actual readings falling into this zone have higher risk to be abnormal ones.

All test results of the healthy subjects tested in a special epidemiologic study were analyzed by separate gender and age groups. For example, all test results of all males of age 30 were put in one group. Predicted values for each HRV parameter were calculated as described above. Then all parameter values (in this example—mean heart rate) were grouped around the predicted values. 5% of the values most deviating from the predicted value are considered as outlying (outside of the normal range). The rest 95% of all values define the normal range.

The ECG recorder considers 15% of the most deviating values among those falling into 95% range as borderline range. Only remaining 80% of all values forms a true normal range used to form an interpretive Autonomic Balance diagram described below. For the subject described in this example a lower borderline level of HF parameter is 2.64. Thus the value shown in the example above falls into a borderline zone.

When using the ECG recorder in FIG. 1 to monitor the dynamics of changes in the autonomic regulatory function or to evaluate the effects of specific factors on this function an important question is usually asked—if the changes in a measured parameter are considered significant or are result of normal variation of the random process. This question is answered based on assessment of the reproducibility and repeatability of the measured parameter. Reproducibility is a variance of a parameter being repeatedly measured in the same subject within a limited time frame. Repeatability reflects natural variance of a specific parameter in the same subject observed during a long period of time (several weeks).

HRV parameters significantly depend on current condition of the subject at a time of testing. Thus it is virtually impossible to obtain absolutely identical readings measured at different moments. This means that the reproducibility and repeatability of the test cannot be 100%. High level of reproducibility and repeatability means only qualitative similarity of any two test results obtained from the same individual at substantially similar conditions of both subject and testing environment. When comparing test results, keep in mind that the autonomic nervous system is fairly sensitive to many internal and external factors including various genetically predetermined and transitory factors, health assessment metrics.

The most appropriate way to assess the autonomic nervous system function is to use so-called predicted values defining normal values of specific HRV parameters, which we expect to obtain from a tested individual if we assume that this individual is healthy. Predicted value is a statistically most probable value of the parameter predicted based on correlation between this parameter values, age and gender of healthy individuals. Predicted values are calculated by the formulae created based on a special study obtained readings from a large population of healthy subjects of different ages and gender.

It's well established that baroreceptor strength declines with age. The reason is that the sensitiveness of the body to any external or internal stimulant makes body easily adaptable to new environment and tells about its strong immunity to fight diseases.

An optimal level of the systemic arterial blood pressure is one of the vital physiological parameters determining adequate function of the cardiovascular system. If arterial pressure is too low then brain, heart and other vital organ do not receive an adequate blood supply so their functions may be affected, e.g. low blood supply to the brain would cause dizziness or even fainting. Alternatively too high arterial pressure causes unnecessary workload to the heart and negatively affects vascular system.

An arterial baroreflex is a key mechanism of short-term regulation of arterial blood pressure. Its whole purpose is to sense minute changes in blood pressure and adjust heart rate to compensate changes in blood supply to the vital organ caused by blood pressure changes. Baroreflex function significantly affects body's ability to adequately react to physical, emotional or mental stressors 93, which may cause significant changes in blood pressure. Decreased baroreflex function may be an early sign of developing cardiovascular disorders such as arterial hypertension and poor overall health 91.

The biological clock 92 test involves continued deep breathing following on-screen instructions for about 1 minute. During that time the ECG recorder analyzes reaction of the user's body on deep paced breathing. The more sensitive the body is, the better shape the user is in. This means that the body is capable to react immediately on changes in internal and external environments, which is a good sign of being younger.

During inhalation the chest is expanding and its internal pressure drops leading to a slight drop in blood pressure because large blood vessels inside the chest are stretched when chest is expanded. The baroreflex causes a quick increase in heart rate as described above. During exhalation the chest contracts so its internal pressure rises causing blood pressure to rise as well due to shrinking large blood vessels in the chest. The baroreflex causes a quick decrease in heart rate as described above. This phenomenon is also known as respiratory sinus arrhythmia. Deep breathing causes maximum possible fluctuations in blood pressure, which helps measuring baroreflex function with larger stimuli. It was found that the highest changes in heart rate induced by deep breathing happen when breathing at the rate of about 6 breaths per minute. Measurement of heart rate oscillations when breathing deeply at 6 breaths per minute is a simple yet effective way to measure baroreflex function. The less sensitive baroreflex is the lesser heart rate oscillations occur.

High baroreflex function is a sign of good vascular elasticity and thus ability of the body to efficiently adapt to various physical, emotional and metal factors causing stress 93 and raise of blood pressure. Low baroreflex function typically is a sign of aging process or certain cardiovascular problem causing stiffness or arterial walls.

The time interval between intrinsic ventricular heart contractions changes in response to the body's metabolic need for a change in heart rate and the amount of blood pumped through the circulatory system. For example, during a period of exercise or other activity, a person's intrinsic heart rate will generally increase over a time period of several or many heartbeats. However, even on a beat-to-beat basis, that is, from one heart beat to the next, and without exercise, the time interval between intrinsic heart contractions varies in a normal person. These beat-to-beat variations in intrinsic heart rate are the result of proper regulation by the autonomic nervous system of blood pressure and cardiac output; the absence of such variations indicates a possible deficiency in the regulation being provided by the autonomic nervous system. One method for analyzing HRV involves detecting intrinsic ventricular contractions, and recording the time intervals between these contractions, referred to as the R-R intervals, after filtering out any ectopic contractions (ventricular contractions that are not the result of a normal sinus rhythm). This signal of R-R intervals is typically transformed into the frequency-domain, such as by using fast Fourier transform, so that its spectral frequency components can be analyzed and divided into low and high frequency bands. For example, the low frequency (LF) band can correspond to a frequency (LF range 0.04 Hz to <0.15 Hz, and the high frequency (HF) band can correspond to a frequency range (HF range of 0.15 Hz −0.40 Hz). The HF band of the R-R interval signal is influenced only by the parasympathetic/vagal component of the autonomic nervous system. The LF band of the R-R interval signal is influenced by both the sympathetic and parasympathetic components of the autonomic nervous system. Consequently, the ratio LF/HF is regarded as a good indication of the autonomic balance between sympathetic and parasympathetic/vagal components of the autonomic nervous system. An increase in the LF/HF ratio indicates an increased predominance of the sympathetic component, and a decrease in the LF/HF ratio indicates an increased predominance of the parasympathetic component. For a particular heart rate, the LF/HF ratio is regarded as an indication of patient wellness, with a lower LF/HF ratio indicating a more positive state of cardiovascular health. A spectral analysis of the frequency components of the R-R interval signal can be performed using a FFT (or other parametric transformation, such as auto-regression) technique from the time domain into the frequency domain. The LF/HF ratio will be used to provide the WM. The LF/HF will be compared to standardized numbers to approximate wellness trends for users.

In another embodiment, the WM may be performed by time domain measurements of the ECG signal. In a continuous ECG record, each QRS complex is detected, and the so-called normal-to-normal (NN) intervals (that is, all intervals between adjacent QRS complexes resulting from sinus node depolarizations) or the instantaneous heart rate is determined. The simplest variable to calculate is the standard deviation of the NN intervals (SDNN), that is, the square root of variance. Since variance is mathematically equal to total power of spectral analysis, SDNN reflects all the cyclic components responsible for variability in the period of recording. The HRV measurement will be used, along with at least one parameter such as body weight, to provide a more complete picture of health.

There needs to be a balance between hard and easy training and rest both within a single training week and within longer training periods. When a hard training session or training period that causes a significant disturbance in body's homeostasis is followed by sufficient recovery, performance improvements are likely to occur. The importance of sufficient recovery is due to the fact that performance improvements actually occur during recovery from training, not during workouts. Finding a balance between training load and recovery is a key factor in improving athletic performance.

Recovery is an important factor with any training regimen. Usually athletes have several very hard training periods each year, during which both the intensity and volume of training are very high. These kind of overreaching periods are very exhaustive but necessary for elite athletes to further improve their performance. However, performance can improve only if hard training is followed by adequate recovery.

Too hard training without sufficient rest may lead to overtraining, which is characterized by decreased performance and in the worst case also other harmful effects on health. Recovery from overtraining may take from several weeks to months, but it is also possible that an athlete never reaches the same level of performance as before overtraining. Prevention of overtraining is therefore crucial, and is possible by systematic assessment of the athlete's recovery. Recovery is defined as decreased activation in the body during relaxation, rest and/or peaceful working, related to lack of external and internal stress factors when parasympathetic (vagal) activity is great and sympathetic activity is low. Recovery is detected when HR is close to the resting level and HRV is great and regular according to the breathing rhythm. Level of HRV is individual. This must be taken into account when interpreting the measured data since analysis is based on HRV. It is recommended that reference values are measured for both high training load/poor recovery and for low training load/well recovered conditions. These reference values should be updated whenever needed, for example between different training periods if changes appear in ANS function.

Respiration can be an indicator of activity, and can provide an explanation of increased sympathetic tone. For example, it may not be appropriate to change or modify a treatment for modulating autonomic tone due to a detected increase in sympathetic activity attributable to exercise. Respiration measurements can be used to measure Respiratory Sinus Arrhythmia (RSA). RSA is the natural cycle of arrhythmia that occurs through the influence of breathing on the flow of sympathetic and vagus impulses to the sinoatrial node (parasympathetic nervous system—PNS). The rhythm of the heart is primarily under the control of the vagus nerve, which inhibits heart rate and the force of contraction. The vagus nerve activity is impeded and heart rate begins to increase when a breath is inhaled. When exhaled, vagus nerve activity increases and the heart rate begins to decrease. The degree of fluctuation in heart rate is also controlled significantly by regular impulses from the baroreceptors (pressure sensors) in the aorta and carotid arteries. Thus, a measurement of autonomic balance can be provided by correlating heart rate to the respiration cycle. The bigger the RSA, the better it is for heart function and blood pressure. Short and rapid breathing is associated with small RSA, and slow and long breath with larger RSA. Thus, slow and long breath is helpful in supporting heart function and lowering blood pressure.

Instant bio-feedback can be provided to the patient, without the need for any interruption arising from repositioning of the device. The bio-feedback derived from HRV and RSA can be used in the areas of stress reduction, rehabilitation, performance enhancement, Migraines and other headaches, AIDS, Depression or Bipolar Disease, Anxiety, Post-traumatic Stress Syndrome, Attention Deficit Disorder, Fibromyalgia, Hypertension, Post-MI, Angina, Atherosclerosis, Mitral Valve Prolapse Syndrome, Cardiomyopathy, Cardiac Dysrhythmias, Congestive Heart Failure, Acquired Hypothyroidism, Thyroid Disorders, Premature Menopausal Symptoms, Menopausal Syndromes, Sleep Apnea, Asthma and COPD (this list is not meant to be exhaustive).

The cover 33 may comprise of memory to store signals for delayed transmission. Conveniently, an archive memory may be used to store standard bio-data such as standard ECG trace of the user, acquired when the user is healthy. This archived bio-signal may then be sent to distant medical professions, along with contemporary signals, when the user/patient is having a crisis. The cover 33 may be coupled to the cell phone by an internal or external connector which extends from the circuitry of the cover to the microphone or data port of the cell phone to transfer the bio-signal data to the cell phone. Data may be transferred to mobile phone or client device using wired or wireless communication 11 comprising at least one of Bluetooth, Zigbee, WiFi, WiMax, IR, a cellular protocol, amplitude modulation or frequency modulation such as Bluetooth, ANT, zigbee and radio. The client device 10 can be a Personal Digital Assistant (PDA), mobile phone, a telehealth hub, laptop computer, personal computer and server. The cover 33 may be powered by its own internal battery source 15 or may piggyback on the power supply of the mobile device. In another embodiment, the power supply 15 is a rechargeable power supply, and more particularly, a rechargeable battery power supply.

In addition to HRV and RSA the ECG recorder can include and/or connected to other sensors and devices that include blood glucose meter, a pacemaker, a blood pressure monitor, an insulin pump, a pulse oximeter, a holter monitor, an electrocardiograph, an electroencephalograph, a blood alcohol monitor, an alcohol breathalyzer, an alcohol ignition interlock, a respiration monitor, an accelerometer, a skin galvanometer, a thermometer, a patient geo-location device, a scale, an intravenous flow regulator, patient height measuring device, a biochip assay device, a sphygmomanometer, a hazardous chemical agent monitor; an ionizing radiation sensor; a monitor for biological agents, a loop recorder, a spirometer, an event monitor, a prothrombin time (PT) monitor, an international normalized ratio (INR) monitor, a tremor sensor, a defibrillator, or any other medical device.

An application programming interface (API) may be used to access data of many different types. In one implementation, the API may be used for bi-directional communication between the wellness cover device and other medical devices for updating and deleting data and metadata.

Beyond a cover, the ECG recorder in FIG. 1 may also be incorporated into a harness, patch/band aid or glove, communicating with mobile or cell phone or client devices, in order to convey the bio-signal into the telephonic transmission portion of the combined device.

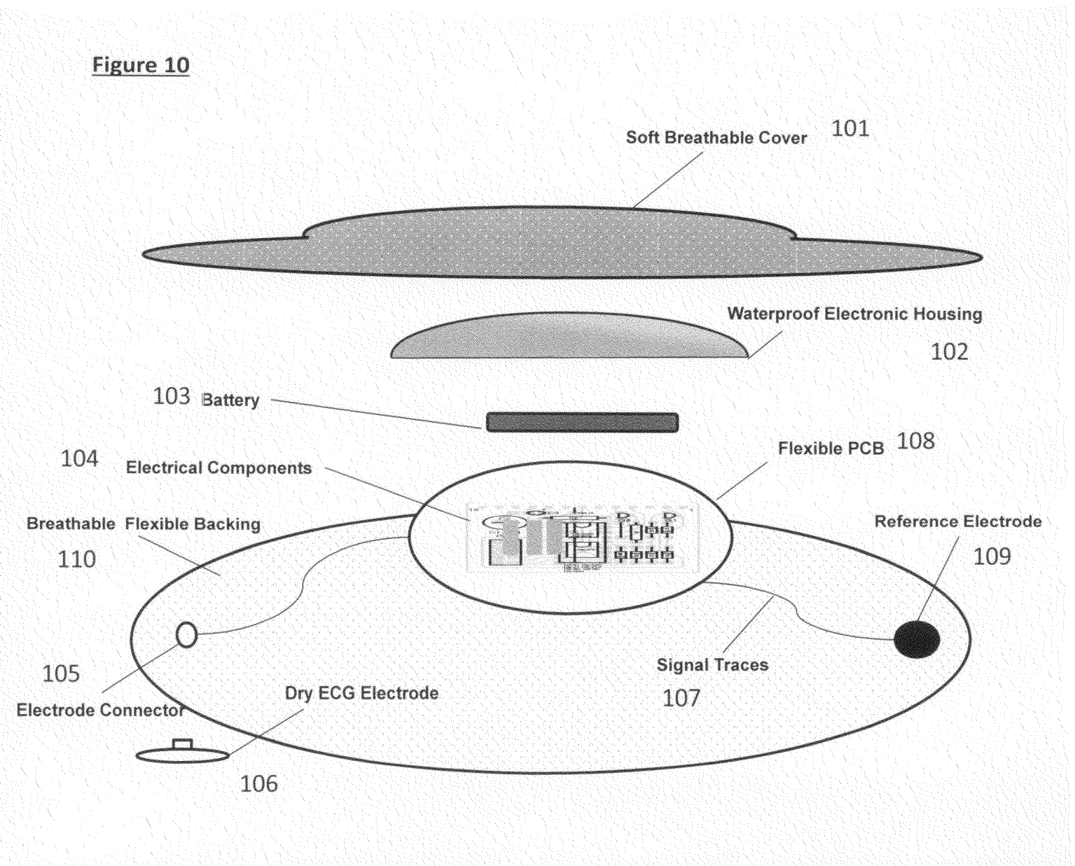

In another embodiment, FIG. 10 shows the ECG recorder in the form of a patch. The patch has a soft breathable cover 101. Underneath the breathable cover 101 is a waterproof electronics housing 102, which houses the electronics 104, battery 103 on a flexible circuit board 108. The patch has a flexible backing 110. Electrical leads 107 are attached onto dry ECG sensors 106, running along the breathable, flexible backing 110, which contains adhesive that adheres to the user.

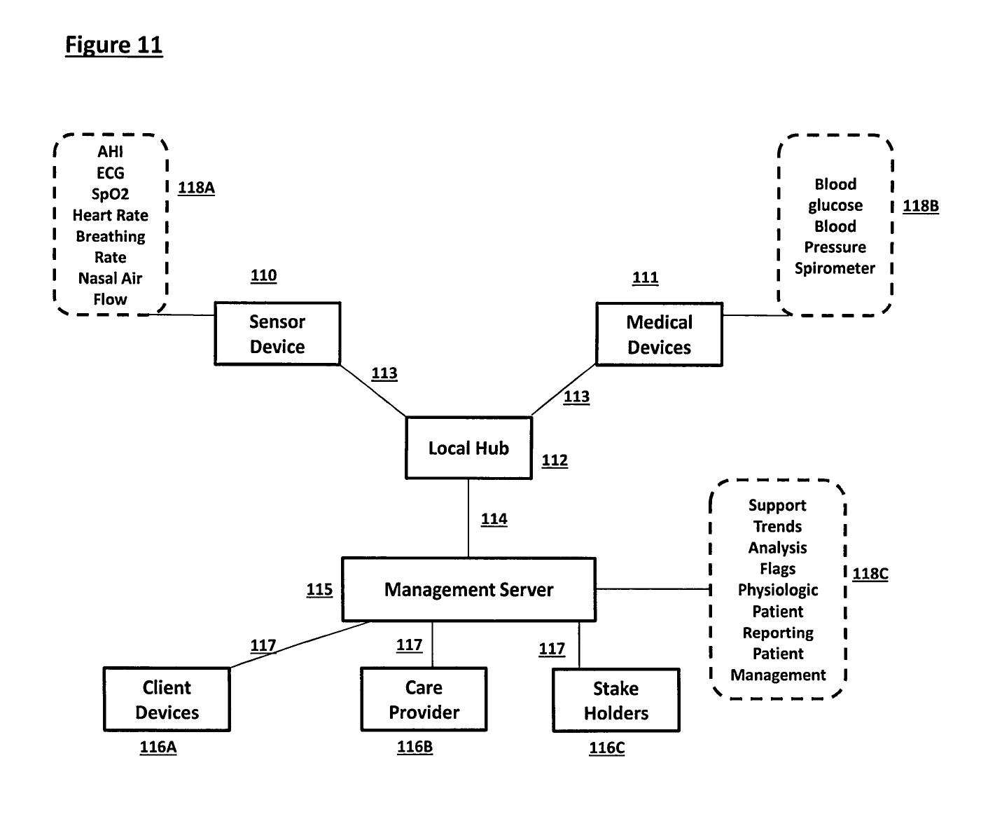

FIG. 11 shows an overall representation of the system's components. Different hardware components are used for data inflow, data outflow (118A,B, C), and information processing/displaying. The wearable patch device or sensor device 110 collects data 118A from the user. The data 118A collected from the sensor device 110 includes blood oxygen saturation (SpO2), heart rate, nasal airflow, EEG, ECG, body temperature, sleep disorder breathing events and severity, sleep staging and body movement data. The data from the sensor device is then transferred to a local hub 112 for display and initial analysis via wireless communication, such as infrared communication, Bluetooth and Zigbee or with a wired connection such as USB (Universal Serial Bus) 113. The local hub can be a mobile device (cell phone, tablets) or personal computer. The data is ultimately transferred to a health management server 115. Other patient medical devices 111 can also collect and transfer third party data to the health management server 115 via the Internet, cellular or a private network 114. These devices 111 include blood glucose meter, a pacemaker, a blood pressure monitor, an insulin pump, a pulse oximeter, a holter monitor, an electrocardiograph, an electroencephalograph, a blood alcohol monitor, an alcohol Breathalyzer, an alcohol ignition interlock, a respiration monitor, an accelerometer, a skin galvanometer, a thermometer, a patient geo-location device, a scale, an intravenous flow regulator, patient height measuring device, a biochip assay device, a sphygmomanometer, a hazardous chemical agent monitor; an ionizing radiation sensor; a monitor for biological agents, a loop recorder, a spirometer, an event monitor, a prothrombin time (PT) monitor, an international normalized ratio (INR) monitor, a tremor sensor, a defibrillator, or any other medical device.

Health Management Server—The health management server is a computer which includes a processing unit, a system memory 4, and a system bus that interconnects various system components, including the system memory to the processing unit. The system memory 44 includes read only memory (ROM) 48 and random access memory (RAM) 50. A basic input/output system 52 (BIOS) is stored in ROM 48.

A number of program modules may be stored on the hard disk, magnetic disk, optical disk, ROM, or RAM. These programs include a server operating system one or more application programs. The operating system can be a Windows-brand operating system such as Windows NT, Windows 7, Windows CE or other form of Windows. The operating system may alternatively be other types, including Macintosh and UNIX-based operating systems.

Application programs (also referred to as “programs”) are used for scoring sleep disordered breathing screener/questionnaire results and determining prevalence of sleep disordered breathing, patient position, fall detection, heart failure and heart arrhythmia. The programs are used to analyze and score data collected from the sensor device 110 and other medical devices 111. The programs analyze data received from the sensor device to send notification of data trends that fall below a pre-established threshold to client device(s), manage patient data and report trends 118C. The management server device sends information118C to client devices116A, care providers 116B and to other stake holder client devices 16C such loved ones and family members via via the Internet, cellular or a private network 117.

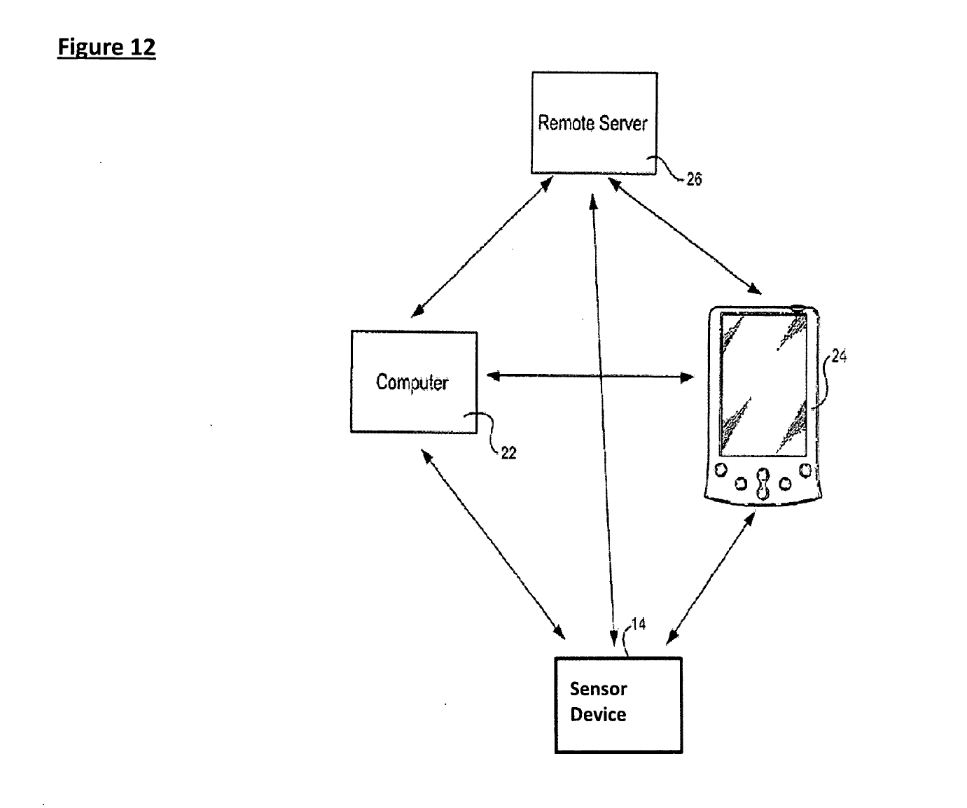

FIG. 12 shows the interaction between the devices. For purposes of this disclosure, local computer 22 and mobile device 24 are defined to include all computing devices, whether portable or stationary. This definition includes, but is not limited to, electronic books, laptop and handheld computers, cellular phones, pagers, desktop computers, personal digital assistant (PDA), telehealth gateway or hub and wearable computers. The sensor devices 14 (referred to as “the devices”) interconnect with the remote health management server 26. For example, the devices 14 may communicate with web applications running on the remote health management server 26 via the Internet or a private network. The devices 14 may include cellular, other wireless or wired communication capability so as to interconnect with the server 26 either continuously or periodically. Communication with the remote health management server 26 may be via the local computer 22 or mobile device 24. The devices 14 may also include some type of memory chip or memory module that may be removed and inserted into the local computer 22 or the mobile device 24 for transfer of data. The devices 14 may communicate with local computer 22 by interconnecting a wire between the computer 22 and the devices 14, or by “docking” the devices 14 into a communications dock associated with computer 22. The devices 14 may also communicate with the computer 22 and a mobile device 24 by wireless communication, such as infrared communication, Bluetooth and Zigbee or with a wired connection such as USB (Universal Serial Bus).

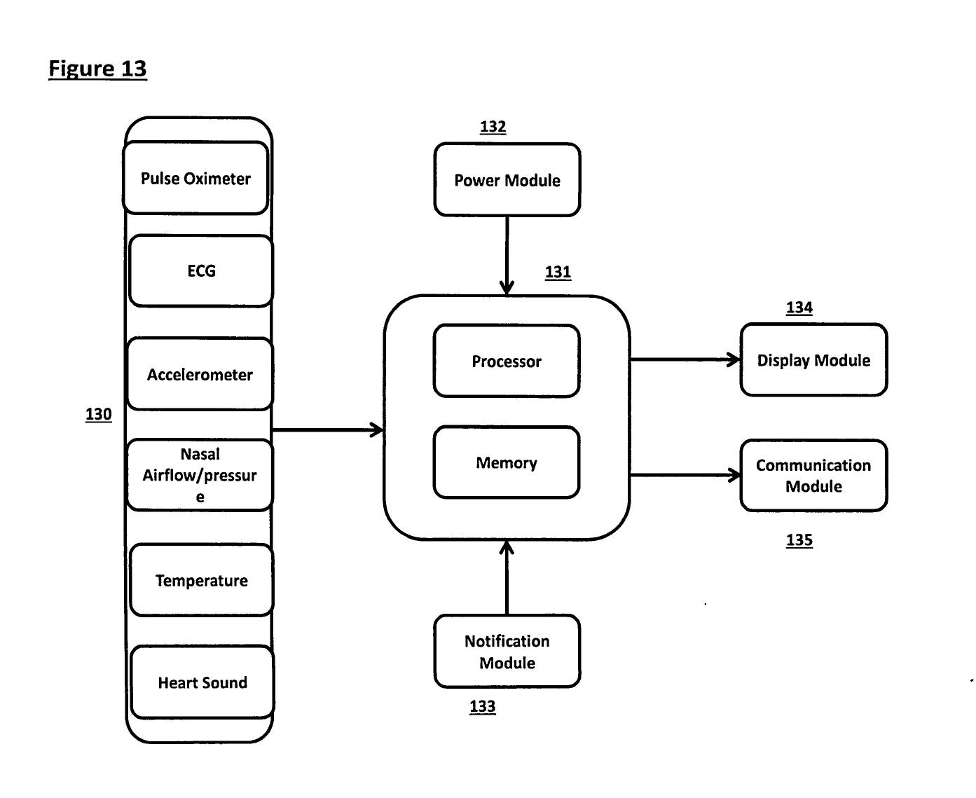

Wearable Sensor Device—Referring to FIG. 13, the wearable sensor device includes a processor, RAM 131, which temporarily provides storage for critical operational data or instructions. Also, alternate embodiments can be provided whereby the contents of the flash memory and the contents of the ROM memory 131 may be interchanged, or whereby the contents may be entirely stored in one type of non-volatile memory and none in the other. Finally, other types of non-volatile memory may be used instead, such as Ferro-electric memory or others. The sensor device further includes a battery system 132 configured to provide a direct current source of power to the sensor device. It also includes a charger that can charge the device via a wall power outlet, USB or some other AC power source. The sensor device can harvest its power from external power source such as body heat. The sensor device includes input /output means interfaced to its programmable circuit 134. The input means may include touch screen, control buttons, or other user-controllable mechanism. The output mechanism 133 can be any type of audio, video, or data interface (LED) or vibration configured to provide information regarding the sensor device to users and devices external to the device.

The sensor device includes a display means 134 for displaying information to a user. The display means includes an LCD 127, CRT, or other screens. Although the sensor device is shown as including a display means, in alternate embodiments a display device is not required. An alarm can be configured to provide various types of audio indications to the user of various conditions detected in the user or the device. These conditions include a health condition detected, such as an abnormally low or high heart rate or blood oxygen level, potential heart failure, body temperature and abnormal heart rhythm. The alarm optionally triggers, based on additional alarm conditions beyond those listed here; the alarms selected generally relate to the type of sensor device implemented and conditions experienced by that device.

A wireless communication module or interface 135 provides a data communication connection from other devices that interface with the sensor device such as a server or other generalized computing system. The communication module or interface is a piece of hardware that is connected to the programmable circuit of the sensor device, and sends and receives data from the other sensor devices. In various embodiments, the communication interface can communicate with various wireless communication protocols such as Bluetooth, and infrared. Other wireless communication protocols such as ZigBee and IEEE 802.11 and cellular wireless can be use alternatively. In another embodiment the communication interface can be wired such as USB (not shown).

The sensor device can also be proximal to the patient's person. The signals 130 can be ECG, EEG, actigraphy, pulse oximetry (heart rate and blood oxygen) 109, skin temperature, nasal airflow and heart sound (from piezoelectric contact microphone with piezo film combined with a low-noise electronic preamplifier to provide a unique sound or vibration pickup with buffered output, from small vibration of the chest picked up by an accelerometer, similar to U.S. Pat. No. 6,650,940 B1, specification is incorporated).

Alternatively, the heart sound can also be acquired using an accelerometer. The propagation path of the higher frequency (>1 Hz) primary heart sounds to the chest is modulated through movements of the chest wall. This is due to the fact that the distance between the chest-worn sensor 110 and the sound source varies directly with respiration-dependent chest wall motion, causing a change in the intensity of the signal detected by the sensor. The change in signal propagation path leads to variable signal attenuation, a modulation of power or amplitude, and is picked up by the sensor 110. In addition, the amplitude of S1 and S2 sounds are themselves modulated throughout the respiratory cycle as stroke volume and pressures are affected: higher pressure differences through the valves lead to faster and stronger impacts and decelerations at valve closure and thus louder sounds. These metrics, occurring at the beat locations in time (hence unevenly sampled), are then interpolated (resampled) and low-pass filtered to generate a respiration trace based on the S1/S2 amplitude.

Respiration modulates the timing of the primary heart sounds S1 and S2 in subtle and indirect ways. Inspiration decreases pleural pressure, and applies pressure on the systemic venous and arterial system. Increased pressure on the venous system increases venous return and pre-load in the right ventricle. The longer filling time results in delayed S1. In the left ventricles, the smaller pre-load (due to interactions between the two ventricles) and higher after-load result in a shorter systole, and an earlier S2. These two effects result in a shortening of the S1-S2 interval during inspiration, and a widening during expiration.

Several metrics heart sounds amplitude can be used to quantify the heart sound amplitudes and their modulation, including but not limited to, maximum absolute amplitude, peak-to-peak amplitude, energy of the whole beat (encompassing S1 and S2), of individual components (S1 or S2), or combination of (ratio of S1 and S2 amplitude or energy, to capture opposite changes during the respiratory cycle).

The sensor can also collect physiological data such as ECG via dry sensors, respiration rate from the movement of the thorax using a 3D accelerometer or respiratory sinus arrhythmia from the ECG, skin temperature from an infrared thermometer, heat flow off the body, nasal air flow from an air pressure or airflow sensor via a cannula going to the nostrils, galvanic skin response or GSR, EMG, EEG, EOG, blood pressure, body fat, hydration level, activity level, oxygen consumption, glucose or blood sugar level, body position from an accelerometer, heart rate, blood oxygen level from earlobe or finger pulse oximeter sensor, pressure on muscles or bones, and UV radiation exposure and absorption (this list is not intended to be exhaustive). The data can, according to one embodiment of the present invention, be sent to memory, such as flash memory, where it is stored until uploaded in the manner to be described below. Thus, memory of the sensor device, over time, stores data relating to the individual sleep onset/wake, therapy, efficacy, sleep duration, sleep performance, and sleep efficiency. That data is periodically uploaded from the sensor device to either a telehealth hub or directly to the health management server subsequent processing and presentation to the user.

The sensor device 110 monitors breathing cessation or apnea-hypopnea index (AHI). The combined data such as AHI, SPO2, heart rate and body movement are used to aid the diagnosis of sleep disordered breathing or sleep apnea. The sensor device110 monitors the nasal air pressure or flow of breathing gas. It receives the flow signal and the pressure signal from flow and pressure sensor, respectively, and uses this information to determine when the patient has transitioned from the inspiratory phase to the expiratory phase of the breathing cycle or vice versa, to control the pressure accordingly. The flow of breathing gas and the pressure level thereof are controlled based on the conditions of the patient. The pressure and flow sensors and other sensors, such pulse oximeter (oxygen desaturation percentage from baseline), are used to detect breathing cessation, and whether or not the airway is at least partially obstructed, manifested by conditions such as snoring, apneas, hypopneas, etc. Once an apnea has been detected it can be further classified as either obstructive or central. In obstructive sleep apnea, the walls of soft tissue in upper airway collapse during sleep, obstructing breathing during sleep. In central sleep apnea, the basic neurological controls for breathing rate malfunction and fail to give the signal to inhale, causing an individual to stop breathing without any obstruction of the airway. Sleep apnea severity is correlated with abnormal heart conditions.

The sensor device 110 can be wearable such as an earpiece, eyewear, armband, wristband or forehead. Once the data is stored on the server 33, it is available on a software interface. The sensor device 110 transmits data to different devices (telehealth hub, client devices and health management server) using short-distance wireless Local Area Network applications such as Bluetooth, Wi-Fi (Wireless Fidelity) and cellular technologies such general packet radio service (GPRS), as well as in the 3G systems. The sensor device can also transmit data via wired connection such as USB (Universal Serial Bus).

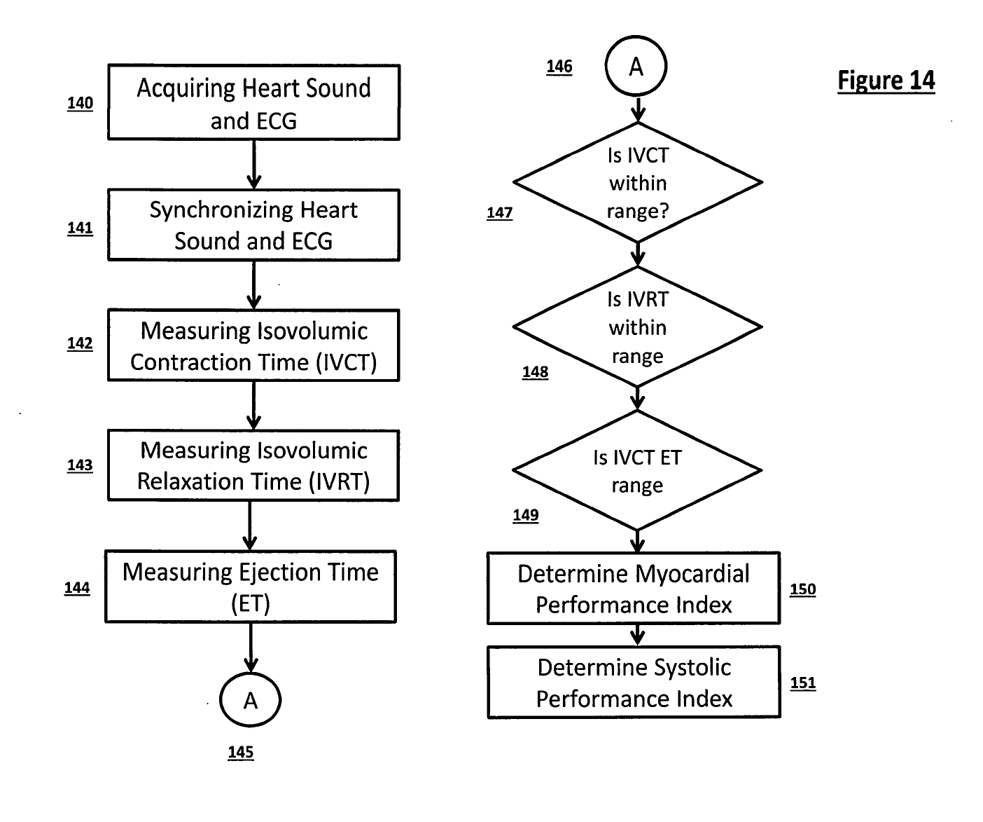

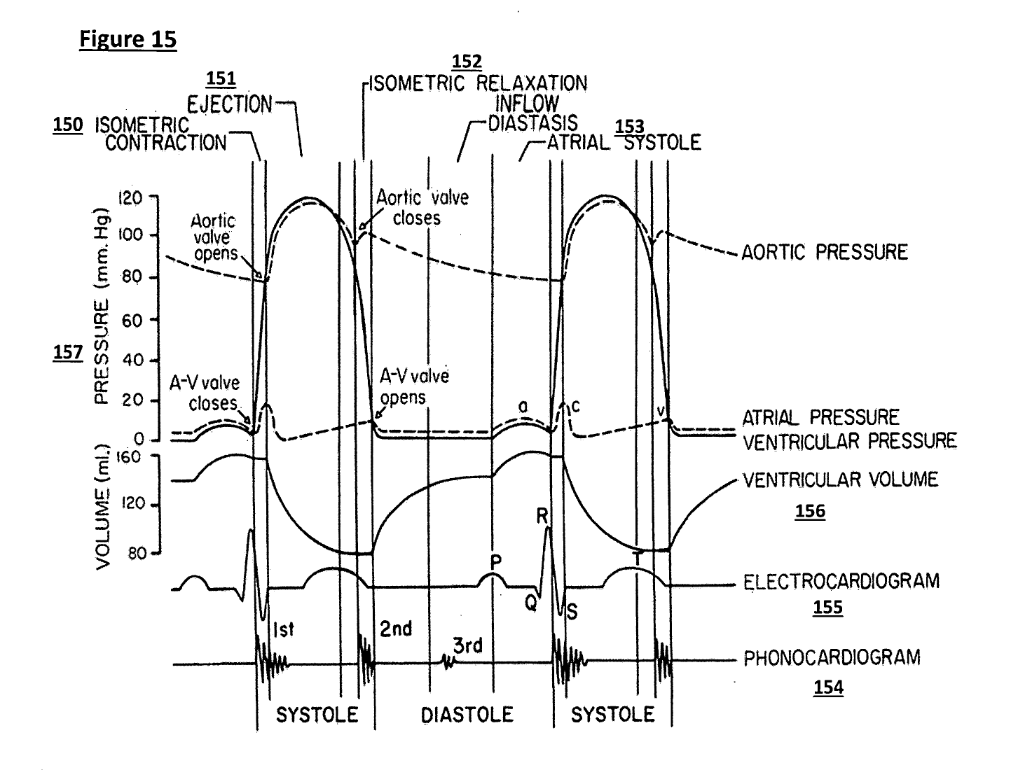

FIG. 14 shows the steps for measuring systolic performance index. Myocardial performance index (MPI) (Tei index) has been regarded as an important parameter in the evaluation of ventricular systolic function in congestive heart failure. MPI is defined as the sum of isovolumic relaxation time (IVRT) and isovolumic contraction time (IVCT) divided by left ventricular ejection time (LVET) ([IVRT+IVCT]/LVET). Systolic performance index (SPI) is the isovolumic or isovolumetric contraction time (the time period between the R peak of the ECG and the first heart sound—S1) divided by ejection period (the time period between the first and second heart sound, S1-S2). Heart sound and ECG are acquired 140 and synchronized 141. From there, an algorithm running either on controller of sensor device, or the telehub device or server measures the isovolumic contraction 142, shown in FIG. 15 item 150. Isovolumetric contraction occurs in early systole, during which the ventricles contract with no corresponding volume change, while contraction causes ventricular pressures to rise sharply. The isovolumetric contraction lasts on average about 0.03 s, but this short period of time is enough to build up a sufficiently high pressure that eventually overcomes that of the aorta and the pulmonary trunk upon opening of the semilunar valves, therefore allowing the correct unidirectional flow of blood. The ejection period is also measured 144, which is time period between the first and second heart sound (S1 and S2) FIG. 15, item 151. Isovolumic relaxation time (IVRT) is measure as well 143, an interval in the cardiac cycle, from the aortic component of the second heart sound, that is, closure of the aortic valve, to onset of filling by opening of the mitral valve FIG. 15 item 152 (the time between S2 and the outset of the T wave). A determination is made as to whether or not those parameters are within range (147-149), after which myocardial 150 and systolic performance 151 indices are determined. MPI is measured when the system detects that the user is in sedentary position.

The ejection fraction (EF) is an important measurement in determining how well your heart is pumping out blood and in diagnosing and tracking heart failure. A significant proportion of patients with heart failure happen to have a normal ventricular ejection fraction at echocardiography during examination. Previously called diastolic heart failure, it is nowadays referred to as heart failure with normal ejection fraction (HFNEF) or HF with preserved ejection fraction. EF measures how much blood the left ventricle pumps out with each contraction. A normal heart's ejection fraction may be between 55 and 70%. An ejection fraction of 60% means that 60% of the total amount of blood in the left ventricle is pushed out with each heartbeat.

Systolic performance index (SPI) was found to have a significant inverse correlation with the value of Left Ventricular Ejection Fraction (LVEF (r=−0.947; P<0.001)), and a significant positive correlation could be seen with the value of Myocardial Performance Index (MPI (r=0.796; P<0.001)). The sensitivity and specificity of SPI value 0.66 for predicting an LVEF value 55% were found to be 100% and 90.9%, respectively, but the positive and inverse predictive values were 91.3% and 100%, respectively (Yilmaz et al. 2007). SPI is measured when the system detects that the user is in a sedentary position.

Other parameters such as third and fourth heart sounds, and the time that it takes the left ventricular to gather enough to close the mitral valve can be obtained (time period between the start of the QRS complex of the ECG to the first heart sound (S1), when the system detects that the user is in sedentary position.

The cardiac cycle FIG. 15 is a period from the beginning of one heart beat to the beginning of the next one. It shows the relationship between the heart's mechanical (phonocardiogram or heart sounds) 154, electrical (ECG) 155, volume 156 and pressure parameters. It consists of two parts:

1. Ventricular contraction called systole.
2. Ventricular relaxation called diastole.
   Each part of the cardiac cycle consists of several phases characterized by either a strong pressure change with constant volume or a volume change with a relatively small change in pressure.

Systole includes:
1. Isovolumic contraction time (IVCT) 150.
2. Ejection Time (ET) 151.
Diastole includes:
3. Isovolumic relaxation time (IVRT 152.
4. Rapid ventricular filling.
5. Slow ventricular filling (diastasis).
6. Atrial contraction 153.

The duration of the cardiac cycle is inversely proportional to the heart rate. The cardiac cycle duration increases with a decrease in the heart rate and on the other it shortens with increasing heart rate. At a normal heart rate of 75 beats per minute, one cardiac cycle lasts 0.8 second. Under resting conditions, systole occupies ⅓ and diastole ⅔ of the cardiac cycle duration. At an increasing heart rate (e.g. during an intensive muscle work), the duration of diastole decreases much more than the duration of systole 154.

Arrhythmia Detection: To identify the ST-segment in an ECG signal, accurate detection of the isoelectric line, the J point and the heart rate are required. The isoelectric line is the baseline of the electrocardiogram, typically measured between the T wave offset and the preceding P wave onset. It indicates no muscular activity in the heart at a particular point of time. This is used as a reference to measure ST-segment deviation. The J point is the inflection point at which the QRS complex changes its direction of propagation. It occurs after the offset of the QRS point within a window of 20-50 samples at the rate of 250 Hz of sampling. Our recognition algorithm involves applying three major functions; Discrete Wavelet Transformation (DWT), Windowing Technique, and Slope Detection; on each ECG cycle 155.

With regard to arrhythmia (cardiac rhythm abnormalities) detection, the system is implemented by the Support Vector Machine (SVM) approach. It is a supervised learning framework which performs classification by constructing an N dimensional hyper-plane that optimally separates the data into two categories (S. R. Gunn, “Support Vector Machines for Classification and Regression,”Technical Report, Dept. of Electronics and Computer Science,University of Southampton, 1998). It is one of the best learning algorithms that gives the flexibility for the choice of the kernel and performs training in less time when compared to other learning algorithms like neural networks. Every heart beat is represented as a row in the data set with its feature values and its class label. SVM aims to find the optimal separating plane and the data points that determine the position and the orientation of the plane are called the support vectors.

The system was designed to classify six major arrhythmia most commonly observed by the cardiologists. The classes include Normal (N), Premature Ventricular Contraction (V), Premature Atrial Contraction (A), Fusion (F), Right Bundle Branch Block (R), and Left Bundle Branch Block (L). We use two types of features to describe each heart beat or one cardiac cycle, i.e. Morphological Features, an DWT Features.

The system uses 12 morphological features, which give the timing, area, energy and correlation information of the signal. The system uses the ST-segment features such as the slope of the ST segment, the ST-deviation measurement and the correlation coefficients of the signal with templates of each class. Each class is represented by a template manually chosen from the MIT-BIH database (MIT-BIH Arrhythmia Database. www.physionet.org/physiobank/) The correlation coefficient lies between 0 and 1; the higher the coefficient is, the more likely it is for the signal to belong to that class.

Twelve morphological features are being used. They are: (i) QS Width, (ii) Pre RR Interval, (iii) Post RR Interval, (iv) QR Width, (v) RS Width, (vi) Mean Power Spectral Density, (vii) Area Under QR, (viii) Area Under RS, (ix) Autocorrelation Value, (x) ST-segment Deviation, (xi) Slope of ST, (xii) Correlation coefficient with class template. The system uses 191 DWT (Discrete Wavelet Transform) coefficients, which are obtained by a 4 level decomposition of the signal with the db2 mother wavelet. These coefficients are based on the 180 samples taken to represent each heartbeat. Six classifiers are trained, each for identifying one arrhythmia type and using all the heart beats in the databank for training and testing. The six single beat classifiers are: Normal (N) vs. All, Ventricular Contraction (V) vs. All, Premature Atrial Contraction (A) vs. All, Left Bundle Branch Block (L) vs. All, Right Bundle Branch Block (R) vs. All and Fusion (F) vs. All.

Each data sample (heart beat) is represented by its class label and all 191+12=203 feature values, including the morphological and DWT features. Since we perform five-fold cross validation experiments, we use four folds of the data for training the classification system. In particular, we train six classifiers, one for identifying a particular type of beat using the one-versus-all scheme, resulting in six binary beat classifiers. After training, the test data set from the remaining fold (i.e., the fold not used for training) is given to the classification system.

The classification is a two stage process. During the first stage, the training data is generated with the features selected. We have divided the ECG beats into five sets of equal number of beats. One out of the five sets is randomly selected and labeled as Test Data Set. The remaining four sets are labeled as Training Data Sets and are passed into the feature extraction module where each beat is represented with the 203 features. The sets thus obtained are used to train the learning algorithm, SVM, which results in a SVM model file containing all the beats which form the hyper plane for classification. In the second stage, the SVM model file and the Test Data Set are given as the input to the SVM classifier tuned to the selected parameters. The output of the classifier gives the prediction of the class for each beat in the Test Data Set.

Regarding sleep disorder breathing (sleep apnea), a single lead ECG, nasal air pressure and a fingertip or earlobe oximeter are being used. The system consists of three modules: a signal processing module, a feature extraction module and a classifier module. In the case of ECG, the measurements with a sampling period of 4 msec are segmented into 1 min epoch and passed through the signal processing module which uses an automated wavelet based analysis to perform de-noising, de-trending and detection of characteristics points: QRS complex, P and T waves. The epoch period can be varied and can be as small as 10s. The morphology changes in the ECG waves allow deriving a signal proportional to the respiratory movement. We use T-wave to extract the surrogate respiratory singal and in cases where T-wave cannot be detected, we use R-wave amplitude. Both time and frequency based parameters are used to extract features. A full feature set of 111 elements and a reduced feature set of 19 elements were used in apnea classification. The full set was used for implementation in the management server 115 and the reduced set was used for implementation in a mobile device 112.

In the case of oximetry signal, again the signal processing module 131 cleans up the signal and detects the characteristic points such as the ODI indices. The Oxygen Desaturation Index (ODI) is the number of times per hour of sleep that the blood's oxygen level drops by 3 percent or more from baseline. ODI is typically measured as part of standard sleep studies, such as a polysomnogram or overnight oximetry. These drops in oxygen levels are called desaturations. ODI is measured by an oximeter, which is a device typically placed on the fingertip that shines a red light through the skin and can estimate the amount of oxygen in the peripheral blood. When breathing becomes disrupted during sleep, as may occur in obstructive sleep apnea, the oxygen levels of the blood may repeatedly fall. These drops are typically associated with collapses of the upper airway, events called apnea or hypopnea. Drops occur less frequently in snoring or upper airway resistance syndrome (UARS), two conditions in which breathing is disturbed, yet to a lesser degree. ODI may be worsened in people with underlying lung disease, and an elevation in ODI may lead to increased oxidative stress in the body that may predispose people to long-term cardiovascular risks, including heart attack, stroke, and memory loss associated with dementia. These consequences are an active area of sleep research. Using time only parameters, a full feature set of 20 elements and a reduced feature set of 3 elements were derived for use in classification. The reduced feature set was used in standalone implementation in smartphones 112 whereas the full feature set was used in computer cloud implementation or management server 115. Support Vector Machine (SVM) classifiers have been used to classify every one minute epoch as an apnea or a non-apnea episode. SVM is a powerful discriminative method for pattern classification.

A nasal cannula is attached to a pressure sensor in the sensor device 110 to extract an index used to assess the severity of sleep apnea based on the total number of complete cessations (apnea) and partial obstructions (hypopnea) of breathing occurring per hour of sleep. These pauses in breathing must last for 10 seconds and are associated with a decrease in oxygenation of the blood. In general, the RDI can be used to classify the severity of disease (mild 5-15, moderate 15-30, and severe greater than 30).

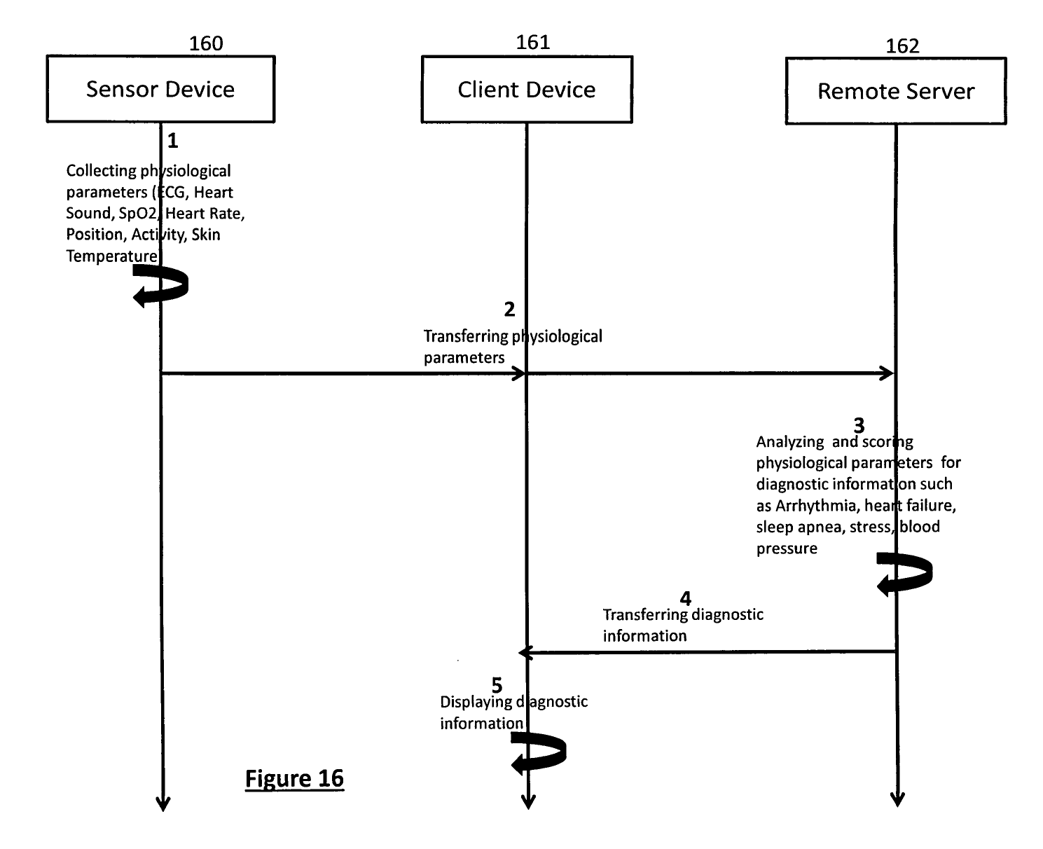

FIG. 16 shows the sequence of an embodiment of the invention. The sensor device 160 collects physiological parameters (ECG, Heart Sound, SpO2, Heart Rate, Position, Activity, Skin Temperature, Nasal airflow). Those parameters are transferred to a client device 161 for initial analysis and display. The parameters are sent to a remote server 162 where they are stored analyzed and scored for diagnostic purposes such as Arrhythmia, heart failure, sleep disordered breathing (sleep apnea), stress, fall detection, body position and sleep hygiene. Those parameters are transferred back to the client device 161 for display and interpretation purposes

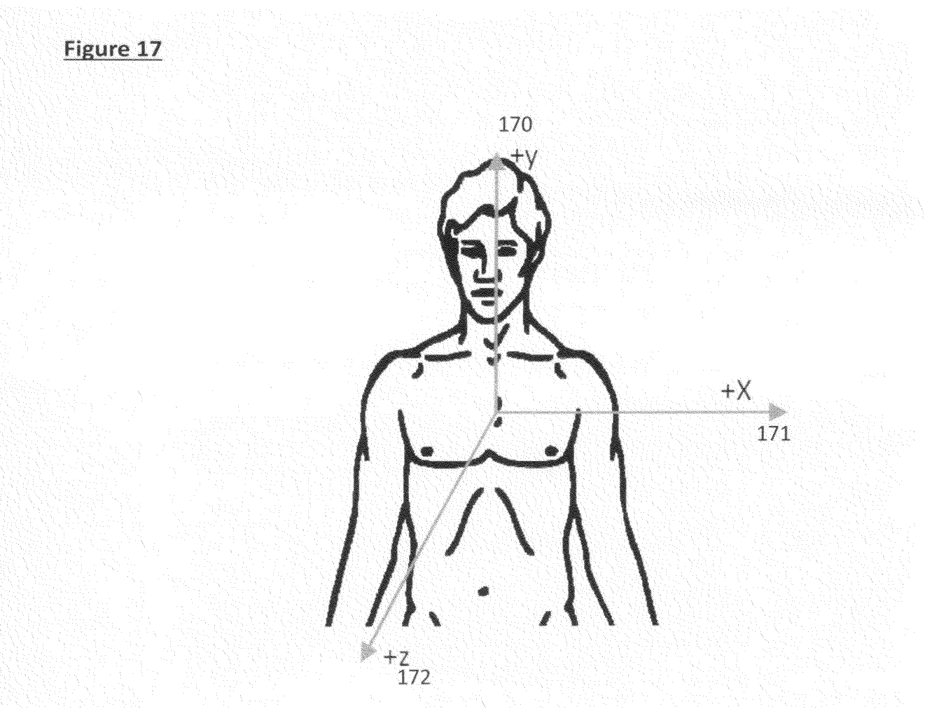

The sensor device in FIG. 13 contains a 3-D accelerometer 130 (such as Freescale MMA8451 Q), that records and stores raw XYZ and “g” values with a sample rate of 12.5 Hz. +X being +1G, Y and Z are defined as indicated in FIG. 17. Movement intensity goes up when the subjects move from one position into the other as expected. Metabolic Equivalent (MET) was used as a bridge to convert movement intensity (motility) (See US cancer research database, google: met walking metabolic equivalent table) into different activity categories. The MET can be multiplied by a user's BMR to make it person specific and turn energy expenditure into a kcal value.

Calculating total energy expenditure of a certain time interval would involve calculating the average MET for the particular time interval and multiplying this average MET with the Basal Metabolic Rate (BMR). Calculating the average MET is done by multiplying the average MET value for each posture class with the time duration of this class within the time interval and dividing this by the total time. The Harris-Benedict equation is still the most used formula for calculating BMR:

For men: (13.75×w)+(5×h)−(6.76×a)+66

For women: (9.56×w)+(1.85×h)−(4.68×a)+655
w=weight in kg
h=height in cm
a=age

The BMR represents the energy expenditure of the person of 24 hours averaging 1 MET. To get to the energy expenditure of a particular time interval, the average MET of this time interval should be multiplied with the BMR, divided by 24 and multiplied by the time interval in hours.

Raw unfiltered measurements are stored on internal FLASH memory.

Firmware algorithm was used to discriminate a human being active vs non-active (not moving or sedentary) states, a human lying vs non-lying, to obtain estimate for energy expenditure in kcal and detect sleep position (sides, supine, prone).

| | Motility [—] | MET [*100] |
|---|---|---|
| Standing Still | 0 | 100 |
| Slow Walking | 17 | 200 |
| Fast Walking | 45 | 400 |
| Slow Running | 127 | 900 |
| Fast Running | 174 | 1200 |

Posture classes are also obtained for the six possible orientations of the body:

1. Upside down

2. Upright

3. Lying down on left side

4. Lying down on right side

5. Lying down on back

6. Lying down on stomach

The algorithm is translated in firmware code. Example firmware codes as follows
//Call this function after the MMA fifo buffer is read into xyzData[ ]
act_process_data(&xyzData[0]);
uint16_t posture[6]; //values represent seconds
uint16_t met_av[6]; //values represent MET * 100
act_calc_results(&posture[0], &met_av[0]); //Call this function before logging a measurement point
//posture[] will contain seconds per posture class of elapsed time interval
//met_avfl will contain average MET per posture class of elapsed time interval

Bedsores, also called pressure sores or pressure ulcers—are injuries to skin and underlying tissue resulting from prolonged pressure on the skin. Bedsores most often develop on skin that covers bony areas of the body, such as the heels, ankles, hips and tailbone. People most at risk of bedsores are those with a medical condition that limits their ability to change positions, requires them to use a wheelchair or confines them to a bed for a long time. Bedsores can develop quickly and are often difficult to treat. Several things can help prevent some bedsores and help with healing.

Bedsores are caused by pressure against the skin that limits blood flow to the skin and nearby tissues. Other factors related to limited mobility can make the skin vulnerable to damage and contribute to the development of pressure sores. Three primary contributing factors are: Sustained pressure. When your skin and the underlying tissues are trapped between bone and a surface such as a wheelchair or a bed, the pressure may be greater than the pressure of the blood flowing in the tiny vessels (capillaries) that deliver oxygen and other nutrients to tissues. Without these essential nutrients, skin cells and tissues are damaged and may eventually die. This kind of pressure tends to happen in areas that aren't well-padded with muscle or fat and that lie over a bone, such as your spine, tailbone, shoulder blades, hips, heels and elbows. Friction. Friction is the resistance to motion. It may occur when the skin is dragged across a surface, such as when you change position or a care provider moves you. The friction may be even greater if the skin is moist. Friction may make fragile skin more vulnerable to injury.

The first step in treating a bedsore is reducing the pressure that caused it. Strategies include the following:

Repositioning. If you have a pressure sore, you need to be repositioned regularly and placed in correct positions. If you use a wheelchair, try shifting your weight every 15 minutes or so. Ask for help with repositioning every hour. If you're confined to a bed, change positions every two hours. If you have enough upper body strength, try repositioning yourself using a device such as a trapeze bar. Caregivers can use bed linens to help lift and reposition you. This can reduce friction and shearing.

For bedsore management, once a lying down posture class is detected, the system includes alarms configured in the mobile device and server (FIG. 11, items 112 and 115) to visually and audibly alert a caregiver of the status of patient conditions.

The present invention has now been described with reference to exemplifying embodiments. However, the invention is not limited to the embodiments described herein. On the contrary, the full extent of the invention is only determined by the scope of the appended claims.

What is claimed is:

1. A method of determining and using a mechanical and electrical footprint of the heart comprising:
a. Acquiring an electrical of the heart;
b. Acquiring a mechanical footprint of the heart;
c. Synchronizing the mechanical and electrical footprints of the heart;
d. Detecting a non-active state;
e. Determining via a processor a time period between R peak of the electrical footprint of the heart and the first heart sound, per the detection of the non-active state;
f. Determining via the processor a time period the first and second heart sound of the mechanical footprint of the heart, per the detection of the non-active state;
g. Calculating systolic performance index (SPI) from the time period between R peak of the electrical footprint of the heart and the first heart sound, and the time period the first and second heart sound of the mechanical footprint of the heart;
h. Correlating left ventricular ejection fraction parameter from the SPI calculation.

2. The method according to claim 1, further comprising of determining the time period between aortic component of the second heart sound to onset of filling by opening of the mitral valve.

3. The method according to claim 1 wherein the heart's electrical and mechanical footprints are transmitted to a mobile device.

4. The method according to claim 1 wherein electrical footprint of the heart is electrocardiogram (ECG).

5. The method according to claim 4 wherein cardiac rhythm abnormalities are derived from the ECG.

6. The method according to claim 1 wherein mechanical footprint of the heart is phonocardiogram.

7. The method according to claim 1 wherein the acquisition of the ECG and phonocardiogram are from a chest patch with dry ECG sensors and piezoelectric microphone, with backing which contains adhesive that adheres to the user.

8. A system for determining and using a mechanical and electrical footprint of the heart comprising:
a. A sensor device with biological sensors, wherein said sensor device contains non-transitory computer readable medium, a processor and software being executed to perform the following:
b. Acquiring an electrical of the heart;
c. Acquiring a mechanical footprint of the heart;
d. Synchronizing the mechanical and electrical footprints of the heart;
e. Detecting a non-active state;
f. Determining a time period between R peak of the electrical footprint of the heart and the first heart sound, per the detection of the non-active state;
g. Determining a time period the first and second heart sound of the mechanical footprint;
h. Calculating systolic performance index (SPI) from the time period between R peak of the electrical footprint of the heart and the first heart sound, and the time period the first and second heart sound of the mechanical footprint of the heart;
i. Correlating left ventricular ejection fraction parameter from the SPI calculation.

9. The system according to claim 7, further comprising of determining the time period between aortic component of the second heart sound to onset of filling by opening of the mitral valve.

10. The system according to claim 7, wherein the myocardial performance index is derived.

11. The system according to claim 7 wherein the heart's electrical and mechanical footprints are transmitted to a mobile device.

12. The system according to claim 7 wherein electrical footprint of the heart is electrocardiogram (ECG).

13. The system according to claim 12 wherein cardiac rhythm abnormalities are derived from the ECG.

14. The system according to claim 7 wherein mechanical footprint of the heart is phonocardiogram.

15. The system according to claim 7 wherein the acquisition of the ECG and phonocardiogram are from a chest patch with dry ECG sensors and piezoelectric microphone, with backing which contains adhesive that adheres to the user.

16. The system according to claim 7, further acquiring pulse oximeter parameter.

17. The system according to claim 7, further acquiring nasal air pressure parameter is acquired.

18. The system according to claim 7, further acquiring sleep disorder breathing parameter.

19. A system for determining and using a mechanical and electrical footprint of the heart comprising:
a. A sensor device with biological sensors, wherein said sensor device contains non-transitory computer readable, a processor and software being executed to do the following:
b. Acquiring an electrical of the heart;
c. Acquiring a mechanical footprint of the heart;
d. Synchronizing the mechanical and electrical footprints of the heart;
e. Detecting a non-active state;
f. Determining a time period between R peak of the electrical footprint of the heart and the first heart sound, per the detection of the non-active state;
g. Determining a time period the first and second heart sound of the mechanical footprint, per the detection of the non-active state;
h. Calculating systolic performance index (SPI) from the time period between R peak of the electrical footprint of the heart and the first heart sound, and the time period the first and second heart sound of the mechanical footprint of the heart;
i. Correlating left ventricular ejection fraction parameter from the SPI calculation;
j. Determining a time period between aortic component of the second heart sound to onset of filling by opening of the mitral valve, per the detection of the non-active state;
k. Deriving myocardial performance index from the R peak of the electrical footprint of the heart and the first heart sound, the time period the first and second heart sound of the mechanical footprint and the time period between aortic component of the second heart sound to onset of filling by opening of the mitral valve, per the detection of the non-active state.
